(19) United States
(12) Patent Application Publication
(10) Pub. No.: US 2025/0188509 A1
(43) Pub. Date: Jun. 12, 2025

(54) METHOD FOR DETECTING AND/OR QUANTIFYING CROSSLINKS FORMED BY TRANSGLUTAMINASES

(71) Applicant: UCB BIOPHARMA SRL, BRUSSELS (BE)

(72) Inventors: PAUL CUTLER, SLOUGH, BERKSHIRE (GB); LIEN BERNICE RIET DEJAGER, BRUSSELS (BE); YVES DUDAL, MUTTENZ (CH); MARK JAIRAJ, SLOUGH, BERKSHIRE (GB)

(21) Appl. No.: 18/718,822

(22) PCT Filed: Dec. 12, 2022
(86) PCT No.: PCT/EP2022/085329

§ 371 Date: Jun. 12, 2024

(30) Foreign Application Priority Data
Dec. 13, 2021 (GB) 2118010.4

(51) Int. Cl. C12Q 1/48; G01N 33/68

(52) U.S. Cl. CPC C12Q 1/48; G01N 33/6848; G01N 2333/91085

(57) ABSTRACT

The present invention relates to methods for detecting crosslinks formed by transglutaminases, including N′N′ Bis(γ-glutamyl)-polyamine and/or ε-(γ-glutamyl)-lysine dipeptide/isopeptide crosslinks in a sample, comprising the steps of digesting proteins present in the biological sample using enzymes immobilised on beads, and detecting said crosslinks in the biological sample by liquid chromatography-tandem mass spectrometry (LC-MS/MS). The methods also allow for determining the activity of transglutaminases, diagnosing diseases associated with transglutaminase activity and assessing treatments.

### CROSS-REFERENCE TO RELATED APPLICATION

This application is the U.S. national stage application of International Patent Application No. PCT/EP2022/085329, filed Dec. 12, 2022.

### REFERENCE TO SEQUENCE LISTING

The Sequence Listing for this application is labeled “Seq-List-replace.xml” which was created on Aug. 12, 2024 and is 59,261 bytes. The entire content of the sequence listing is incorporated herein by reference in its entirety.

### FIELD OF THE INVENTION

The present invention relates to methods for detecting crosslinks formed by transglutaminases, including N′N′ bis (γ-glutamyl)-polyamine and/or ε-(γ-glutamyl)-lysine dipeptide/isopeptide crosslinks in a sample, comprising the steps of digesting proteins present in the biological sample using enzymes immobilised on beads, and detecting said crosslinks in the biological sample by liquid chromatography-tandem mass spectrometry (LC-MS/MS). The methods also allow for determining the activity of transglutaminases, diagnosing diseases associated with transglutaminase activity and assessing treatments.

### INTRODUCTION

The ability to detect and quantify crosslinks, including N′N′ bis (γ-glutamyl)-polyamine and/or ε-(γ-glutamyl)-lysine (epsilon(gamma-glutamyl)-lysine) dipeptide/isopeptide crosslinks (See FIG. 1), in biological samples, for example as a measure of the activity of transglutaminases (TGs) such as transglutaminase 2 (TG2), has significant analytical and clinical potential. However, this has remained a significant challenge.

Transglutaminases comprise a family of structurally related enzymes that catalyse the calcium-dependent post-translational modification of a range of proteins via transamidation reactions to form isopeptide bonds that result in protein crosslinking, for example between glutamate and lysine residues. Thus, TGs form (γ-glutamyl)-polyamine bonds (such as N′N′ bis (γ-glutamyl)-polyamine dipeptide crosslinks and ε-(γ-glutamyl)-lysine isopeptide bonds), within and between proteins. TG2 is one member of this family in humans and is expressed in all cellular compartments and secreted. TG2 has been shown to play a significant role in many human diseases, including fibroproliferative diseases, such as progressive kidney disease, and liver cirrhosis. Fibrosis is a process relating to aberrant wound healing. TG2 contributes to profibrotic events, in part due to it its role in crosslinking extracellular matrix proteins, which makes the proteins increasingly protease resistant. Other human TGs include Factor XIIIa, which is involved in the formation of fibrin clot; TG1 (keratinocyte TG), TG3 (epidermal TG) and TG5, which are involved in terminal differentiation of the keratinocyte in skin; TG4 (prostate TG2), which is thought to have a role in stabilisation of the vascular wall; and TG6, which is involved in neuron function and membrane integrity, and inhibition of TG6 causes ataxia.

Several methods have been considered for detection of the crosslink in tissue and other biological samples, but most have been flawed in some way. TGs can crosslink a wide range of substrate proteins at multiple sites both within and between proteins. This heterogeneity of the cross link has hampered efforts to generate an antibody suitable for detection of the crosslink. Proteomic approaches, for example using LC-MS/MS, have also not progressed. These methods require defined sequence information and the heterogeneity of the crosslink means that each individual peptide containing the crosslink is present in extremely low amounts, hindering detection.

Most prior art approaches for detecting crosslinks rely on amino acid analysis. These methods are based on hydrolysis of the proteins down to the composite amino acids, leaving the crosslinked dipeptides/isopeptides, such as ε-(γ-glutamyl)lysine (γ-Glu-ε-Lys) and/or N′N′ bis (γ-glutamyl)-polyamine isopeptide/dipeptide, intact. The isopeptide/dipeptide signal is then the composite signal from all crosslinks in proteins in the biological sample. Since ε-(γ-glutamyl)-lysine dipeptides or N′N′ bis (γ-glutamyl)-polyamine dipeptides are not stable under extreme acid hydrolysis, enzymatic digestion approaches have been used. Typical methods subjected protein samples to exhaustive serial digestion using proteases which will not cleave the (γ-glutamyl)-polyamine or ε-(γ-glutamyl)-lysine dipeptide bond, such as trypsin and subtilisin, followed by detection of the dipeptide using classical amino acid analysis with the chromatography optimised to resolve the minor dipeptide peak from the major peaks of the common amino acids. However, these methods are subject to major limitations including the inability to resolve the dipeptide from the common amino acids, preventing accurate quantification. Furthermore, complete digestion required milligram amounts of proteases, often exceeding the total protein substrate in a given sample. The enzymes must be added sequentially to enable optimal kinetics and to adapt the buffer conditions, meaning that each protease may be a substrate for the next protease. This significantly altered the reaction kinetics, the digestion efficiency and the resulting chromatogram, again preventing accurate quantification.

The present invention uses an approach based on the principle of complete digestion and release of the crosslinked dipeptides/isopeptides, such as N′N′ bis (γ-glutamyl)-polyamine dipeptides and/or ε-(γ-glutamyl)-lysine dipeptides, but addresses the major disadvantages of the prior art. The present inventors have used improved biological sample preparation, innovative immobilised enzyme technology, allowing for efficient digestion without enzyme contamination, and LC-MS/MS detection based on multiple reaction monitoring, in order to provide a highly specific, sensitive and reproducible assay. This approach represents a significant advance in the ability to detect and quantify crosslinks formed by transglutaminases, including N′N′ bis (γ-glutamyl)-polyamine and/or ε-(γ-glutamyl)-lysine dipeptide/isopeptide crosslinks in biological samples.

### SUMMARY OF THE INVENTION

The present invention provides a method for detecting crosslinks formed by transglutaminases (TGs) in a biological sample, the method comprising the steps of: a) digesting proteins present in the biological sample using enzymes immobilised on beads, and b) detecting crosslinks formed by TGs in the biological sample by liquid chromatography-tandem mass spectrometry (LC-MS/MS). Also provided is a method for determining the activity of TGs, preferably transglutaminase 2 (TG2), in a subject, the method comprising the steps of: a) digesting proteins present in a biological sample from the subject using enzymes immobilised on beads, and b) detecting crosslinks formed by TGs in the biological sample by liquid chromatography-tandem mass spectrometry (LC-MS/MS).

In some exemplary, but not limiting, instances, the method may comprise the steps of: a) enriching the proteins present in the biological sample by precipitating the proteins by treatment with trichloroacetic acid and separating the precipitated proteins by centrifugation, b) digesting the proteins using enzymes immobilised on beads, wherein the enzymes comprise proteinase K, pronase, prolidase, leucin aminopeptidase and carboxypeptidase Y, to produce a mixture comprising free amino acids and dipeptides/isopeptides, and c) detecting and/or quantifying the amount of crosslinks formed by TGs in the biological sample by LC-MS/MS. Preferably the crosslinks formed by TGs comprise N′N′ bis (γ-glutamyl)-polyamine dipeptides/isopeptides and/or ε-(γ-glutamyl)-lysine dipeptides/isopeptides.

Also provided herein is an in vitro method for (i) diagnosing a disease in a subject, or (ii) stratifying the severity of a disease in a subject, the method comprising the steps of: a) digesting proteins present in the biological sample from the subject using enzymes immobilised on beads, and b) detecting crosslinks formed by TGs in the biological sample by liquid chromatography-tandem mass spectrometry (LC-MS/MS). The presence of crosslinks formed by TGs in the biological sample may indicate that the subject has a disease. Typically, the higher the amount/number of crosslinks formed by TGs in the biological sample the more severe the disease and/or the poorer the prognosis of the disease.

Also provided herein is an in vitro method for monitoring the progression of a disease in a subject, the method comprising the steps of: a) digesting proteins present in a biological sample from the subject using enzymes immobilised on beads, and b) detecting crosslinks formed by TGs in the biological sample by liquid chromatography-tandem mass spectrometry (LC-MS/MS); and c) comparing the amount of crosslinks formed by TGs in the biological sample with the amount of crosslinks formed by TGs in a previous sample from the patient or with a control value. Further provided is a method for determining a subject's response to a treatment, the method comprising the steps of: a) digesting proteins present in a first biological sample from the subject using enzymes immobilised on beads, and b) detecting crosslinks formed by TGs in the biological sample by liquid chromatography-tandem mass spectrometry (LC-MS/MS); and c) comparing the amount of crosslinks formed by TGs in the biological sample with the amount of crosslinks formed by TGs in a biological sample from the patient prior to treatment, or at an earlier stage in treatment, or with a control value. Further provided is a method for determining the effect of an agent on TG activity, the method comprising the steps of: a) digesting proteins present in a biological sample from a subject administered the agent using enzymes immobilised on beads, and b) detecting crosslinks formed by TGs in the biological sample by liquid chromatography-tandem mass spectrometry (LC-MS/MS).

Preferably, the crosslinks formed by TGs comprise N′N′ bis (γ-glutamyl)-polyamine dipeptides/isopeptides and/or ε-(γ-glutamyl)-lysine dipeptides/isopeptides.

In any of the methods of the invention, the disease may be any disease associated with elevated TG activity, such as elevated TG2 activity, optionally wherein the disease is fibrosis, a fibrotic disease or a fibrosis-related disease such as chronic kidney disease, progressive kidney disease, pulmonary fibrosis, systemic sclerosis, liver cirrhosis, cardiovascular disease, idiopathic hypertrophic cardiomyopathy, renal fibrosis, primary glomerulonephritis, liver cirrhosis, chronic allograft injury (CAI), post-transplant renal fibrosis or chronic allograft nephropathy. In any of the methods of the invention, the treatment or agent may be an inhibitor of TG, such as an anti-TG antibody.

### DESCRIPTION OF THE FIGURES

FIG. 1. The formation N′N′ bis (γ-glutamyl)-polyamine and ε-(γ-glutamyl)-lysine crosslinks by transglutaminases, such as transglutaminase 2 (TG2).

FIGS. 2A-2B. Schematics illustrating core steps of the methods. (FIG. 2A) Proteins may be enriched from urine samples using trichloroacetic acid (TCA) precipitation methods, proteins are subjected to enzymatic digestion, and resulting amino acids and dipeptides are analysed by LC-MS/MS. (FIG. 2B) TG activity results in intra- and inter-molecular ε-(γ-glutamyl)-lysine (γ-Glu-ε-Lys) crosslinks in proteins. Full enzymatic digestion cleaves the peptide bonds to produce single amino acids and γ-Glu-ε-Lys dipeptides, which are subsequently analysed by LC-MS/MS.

FIGS. 3A-3C. Characterisation of sample preparation. (FIG. 3A) Digestion reproducibility is good (CV<15%). (FIG. 3B) Digestion efficiency is high (>80%) as estimated in comparison to acid hydrolysis. (FIG. 3C) Digestion efficiency is not impacted by protein content. Correlation between the γ-Glu-ε-Lys dipeptide concentration and the concentration of protein submitted to proteolytic digestion. Error bars denote standard deviation (n=3).

FIGS. 4A-4B. Analysis of γ-Glu-ε-Lys dipeptides by LC-MS/MS. (FIG. 4A) A spectrum of the MS/MS analysis of the cross link, indicating the key transitions observed. (FIG. 4B) LC-MS/MS specifically detects the dipeptide. EK: Glutamyl-lysine and KE: Lysyl-glutamate (known isomers of γ-Glu-ε-Lys).

FIGS. 5A-5B. Screening of disease-state and healthy urine samples. γ-Glu-ε-Lys levels measured in urine samples from disease-state and healthy urine samples, expressed as (FIG. 5A) ng γ-Glu-ε-Lys/mL urine as read-out from the LC-MS/MS measurement, or as (FIG. 5B) ng γ-Glu-ε-Lys/mg protein present in urine sample, used as the reported value.

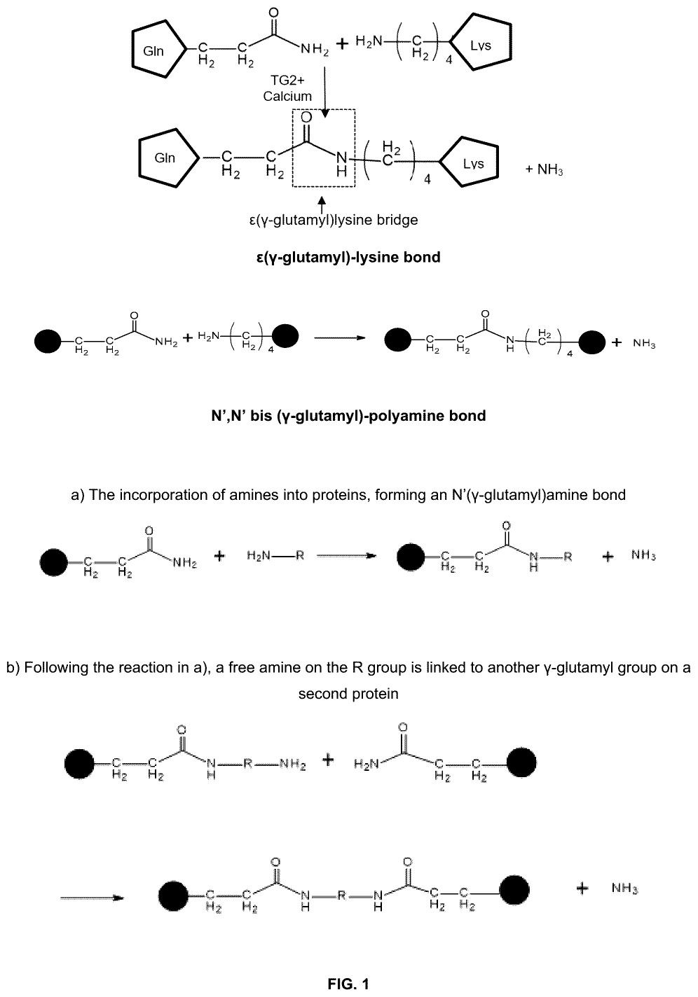

### DETAILED DESCRIPTION

The entire document is intended to be related as a unified disclosure, and it should be understood that all combinations of features described herein are contemplated, even if the combination of features are not found together in the same sentence, or paragraph, or section of this document. With respect to aspects of the invention described or claimed with “a” or “an”, it should be understood that these terms mean “one or more” unless context unambiguously requires a more restricted meaning.

The term “or” should be understood to encompass items in the alternative or together, unless context unambiguously requires otherwise. If aspects of the invention are described as “comprising” a feature, embodiments also are contemplated “consisting of” or “consisting essentially of” the feature.

All publications, patents and patent applications cited herein, whether supra or infra, are hereby incorporated by reference in their entirety.

The present inventors have developed a highly sensitive assay to detect and quantify the amount (alternatively the number; number and/or amount can be used indifferently through this document) of isopeptide bonds (also herein called crosslinks) formed by TGs, such as N′N′ is (γ-glutamyl)-polyamine dipeptide/isopeptide bonds or crosslinks and/or ε-(γ-glutamyl)-lysine dipeptide/isopeptide bonds or crosslinks, in a biological sample. The methods of the invention are capable of specifically, reliably and sensitively detecting the activity of TGs (in particular TG2) in a patient through the detection and quantification of dipeptides/isopeptides formed by TGs, such as N′N′ bis (γ-glutamyl)-polyamine dipeptides/isopeptides and/or ε-(γ-glutamyl)-lysine dipeptides/isopeptides, in a biological sample from the patient. This allows for accurate and sensitive diagnosis of diseases or disorders associated with aberrant activity of TGs, which for TG2 includes fibrosis and other fibrotic disorders. The methods also provide a specific, sensitive, reliable and accurate assay for assessing the target engagement and pharmacodynamics of therapeutic agents targeting the TG pathways, such as therapeutic anti-TG2 antibodies. Dipeptides, isopeptides, dipeptide bonds, isopeptide or crosslinks are herein used indifferently.

The methods and assays described herein overcome the challenge of selectively detecting and quantifying crosslinks formed by transglutaminases, including N′N′ bis (γ-glutamyl)-polyamine dipeptide/isopeptide bonds or crosslinks and/or ε-(γ-glutamyl)-lysine dipeptide/isopeptide bonds or crosslinks. The surprising specificity, selectivity and sensitivity of the methods and assays was achieved by using a combination of (i) on-bead enzymatic digestion and (ii) fast and specific LC-MS/MS detection. The specificity, selectivity and sensitivity of the methods and assays can be further enhanced by efficient biological sample preparation. In particular, the following steps provide particularly improved specificity, selectivity and sensitivity:
Preliminary purification to remove impurities, which dramatically reduces background signal from contaminants in the biological sample.
Use of enzymes linked to protective beads, which allows for specific digestion (producing a mixture comprising free amino acids and crosslinks) and considerably reduces the background caused by proteases digestion contamination.
Use of LC-MS/MS as the detection tool, which allows for highly specific, accurate, precise and quantitative detection.

The methods of the present invention are for detecting and/or quantifying, preferably detecting and quantifying, crosslinks formed by transglutaminases (TGs). Crosslinks formed by TGs typically comprise N′N′ bis (γ-glutamyl)-polyamine dipeptides/isopeptides and/or ε-(γ-glutamyl)-lysine dipeptides/isopeptides, preferably ε-(γ-glutamyl)-lysine dipeptides. In preferred aspects, the methods of the present invention are for detecting and/or quantifying, preferably detecting and quantifying, N′N′ bis (γ-glutamyl)-polyamine dipeptide/isopeptide bonds or crosslinks and/or ε-(γ-glutamyl)-lysine dipeptide/isopeptide bonds or crosslinks. Typically, the methods of the present invention are for detecting and/or quantifying, preferably detecting and quantifying, N′N′ bis (γ-glutamyl)-polyamine dipeptides/isopeptides and/or ε-(γ-glutamyl)-lysine dipeptides/isopeptides. In most preferred aspects, the methods of the present invention are for detecting and quantifying N′N′ bis (γ-glutamyl)-polyamine dipeptides/isopeptides and/or ε-(γ-glutamyl)-lysine dipeptides, which are indicative of TG crosslinking activity. In some aspects, the methods of the present invention are for detecting and quantifying N′N′ bis (γ-glutamyl)-polyamine dipeptides/isopeptides and ε-(γ-glutamyl)-lysine dipeptides. In some aspects, the methods of the present invention are for detecting and quantifying ε-(γ-glutamyl)-lysine dipeptides.

The methods of the present invention for detecting and quantifying crosslinks formed by transglutaminases, including N′N′ bis (γ-glutamyl)-polyamine dipeptides/isopeptides and/or ε-(γ-glutamyl)-lysine dipeptides/isopeptides, in biological samples with high specificity represent a marked improvement over the methods of the prior art, which did not provide the necessary accuracy or sensitivity, especially needed for clinical use.

The methods of the present invention typically comprise the following core steps:
a) digesting proteins present in a biological sample using enzymes immobilised on beads (thereby producing a mixture comprising free amino acids and crosslinks), and
b) detecting crosslinks formed by transglutaminases, including N′N′ bis (γ-glutamyl)-polyamine dipeptides/isopeptides and/or ε-(γ-glutamyl)-lysine dipeptides/isopeptides, in the biological sample by liquid chromatography-tandem mass spectrometry (LC-MS/MS).

The basic method of the invention has a number of different applications. Thus, the present invention provides:
A. a method for detecting crosslinks formed by transglutaminases, particularly for detecting N′N′ bis (γ-glutamyl)-polyamine dipeptides/isopeptides and/or ε-(γ-glutamyl)-lysine dipeptides/isopeptides, in a biological sample,
B. a method for determining the activity of TGs, such as TG2, in a subject;
C. a method for diagnosing a disease in a subject;
D. a method for stratifying the severity of a disease in a subject;
E. a method for monitoring the progression of a disease in a subject;
F. a method for determining a subject's response to a treatment; or
G. a method for determining the effect of an agent on TG activity, such as TG2 activity.

The methods may further comprise obtaining a biological sample from a subject. The methods may further comprise enriching or purifying the proteins present in the biological sample, which is described in more detail below. The methods of the invention are typically performed in vitro.

The methods of the invention may be used to detect crosslinks formed by transglutaminases, preferably to detect N′N′ bis (γ-glutamyl)-polyamine dipeptides/isopeptides and/or ε-(γ-glutamyl)-lysine dipeptides/isopeptides, in any biological sample. In some preferred aspects, the biological sample is urine. The formation of crosslinks particularly such as N′N′ bis (γ-glutamyl)-polyamine dipeptides/isopeptides and/or ε-(γ-glutamyl)-lysine dipeptides/isopeptides, is catalysed by transglutaminases (TGs), including Factor XIIIa, TG1, TG2, TG3, TG5, TG4, TG6, and TG7, and so the methods described herein may be used as an assay for TG activity, such as TG2 activity. Typically, the methods of the invention comprise detecting crosslinks formed by TGs in a biological sample from a subject. Thus, the methods of the invention typically comprise detecting N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in a biological sample from a subject. The presence of N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in the biological sample is indicative of TG activity, such as TG2 activity, in the subject (i.e., in vivo). Thus, the in vitro methods of the invention provide a read-out of in vivo enzyme activity. Accordingly, the methods can also provide insight into disease states in the subject. All of the methods of the invention rely on detecting N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides.

The methods of the invention may be used to detect crosslinks formed by TGs in any sample, preferably a biological sample. Thus, the methods of the invention may be used to detect detecting N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides in any sample, preferably a biological sample. The biological sample may be selected from the group consisting of: urine, blood, plasma, serum, red blood cells, tissue (e.g., kidney tissue, lung tissue, heart tissue, etc.), saliva, placental tissue, bone marrow, breast milk, bronchoalveolar lavage, faeces, pleural fluid, synovial fluid, and semen. Typically, the biological sample is urine or tissue (e.g., kidney tissue, lung tissue, heart tissue, etc.), preferably urine. The biological sample may be or have been obtained or isolated from a subject. Methods for obtaining biological samples from a subject are well known in the art. Tissue samples may be obtained for example by biopsy. The subject is typically an animal, preferably a mammal. In some aspects, the subject is a mouse, a rat, a primate, or a human. In preferred aspects, the subject is a primate, most preferably a human. The subject may be a human patient. In some aspects, the subject may have or be suspected of having, a disease associated with TG activity, preferably TG2 activity, as defined further herein.

The biological sample typically needs to be prepared for use in the methods of the invention. The exact method of sample preparation used may vary depending on the type of biological sample. Typically, the protein fraction of the biological sample is enriched (e.g., concentrated and/or partially purified) for use in the methods of the invention. Thus, the methods may comprise enriching the proteins present in the biological sample, prior to digesting the proteins using the enzyme immobilised on beads. The step of enriching may be understood to mean that the protein fraction is concentrated and/or key contaminants that may interfere with subsequent analysis are removed. The step of enriching may be further understood to mean separating the protein fraction of a biological sample from the other components of the biological sample, for example to prepare a composition consisting essentially of a mixture of all of the proteins present in the biological sample, and excluding other components of the biological sample, such as nucleic acids and lipids. In some aspects, enriching the proteins present in the biological sample comprises precipitating the proteins. Typically, precipitating the proteins advantageously allows for enriching the proteins, concentrating the sample and/or, preferably and, removing salts which may inhibit the subsequent enzymatic digestion. The proteins may be precipitated by salting out for example using ammonium sulphate, for example with trichloroacetic acid, by precipitation with miscible solvents for example with ethanol or methanol, or by precipitation with non-ionic hydrophilic polymers, such as dextran and polyethylene glycols. Preferably, enriching the proteins present in the biological sample comprises precipitating the proteins using trichloroacetic acid. Typically, the precipitated proteins are then separated from the remainder of the biological sample, for example by centrifugation. Thus, enriching the proteins present in the biological sample may comprise precipitating the proteins using trichloroacetic acid and separating the precipitated proteins by centrifugation. The separated precipitated proteins are typically hydrated in a suitable buffer (such as a potassium phosphate buffer, typically about pH 7.5 and which may comprise urea) for subsequent analysis and enzyme digestion. Thus, enriching the proteins present in the biological sample may comprise precipitating the proteins using trichloroacetic acid, separating the precipitated proteins by centrifugation, and hydrating or re-suspending the proteins in a buffered solution.

The methods of the invention comprise digesting proteins present in a biological sample using enzymes immobilised on beads. Typically, substantially all of the proteins in the biological sample are digested. A ‘protein’ or ‘polypeptide’ may be understood to mean a linear polymer chain comprised of amino acid monomers linked by covalent peptide bonds. The proteins present in a biological sample may further comprise intermolecular or intramolecular covalent cross-links (e.g., crosslinks, isopeptide bonds). Digesting the proteins may be understood to mean cleaving the peptide bonds in the proteins, to break the proteins down into their constituent amino acids and, if present, any non-digestible crosslinked isopeptides/dipeptides. Protein digestion may be complete or incomplete. Complete protein digestion typically comprises cleaving all peptide bonds in the proteins to produce a mixture of free amino acids and any non-digestible crosslinked isopeptides/dipeptides. The enzymes used in the methods of the invention are capable of cleaving peptide bonds between amino acids in proteins. The enzymes are typically not capable of cleaving crosslinks formed by TGs, such as N′N′ bis (γ-glutamyl)-polyamine dipeptide/isopeptide and/or ε-(γ-glutamyl)-lysine dipeptide/isopeptide, particularly ε-(γ-glutamyl)-lysine bonds. N′N′ bis (γ-glutamyl)-polyamine dipeptide/isopeptide bonds and ε-(γ-glutamyl)-lysine dipeptide/isopeptide bonds are crosslinks (e.g., isopeptide bonds) within or between proteins that covalently link amino acids from two polypeptide chains. A ε-(γ-glutamyl)-lysine bond has the structure:

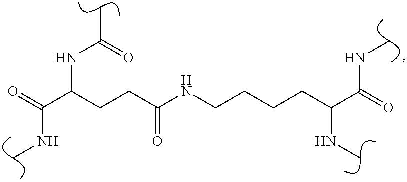 [SMILES: CNC(=O)C(CCCCNC(=O)CCC(NC(C)=O)C(=O)NC)NC]

wherein the curved lines indicate peptide bonds within a linear polypeptide chain. The formation of N′N′ bis (γ-glutamyl)-polyamine dipeptide/isopeptide bonds and ε-(γ-glutamyl)-lysine dipeptide/isopeptide bonds, particularly ε-(γ-glutamyl)-lysine bonds, is catalysed by TGs. N′N′ bis (γ-glutamyl)-polyamine dipeptide/isopeptide bonds and/or ε-(γ-glutamyl)-lysine dipeptide/isopeptide bonds, particularly ε-(γ-glutamyl)-lysine bonds, may be formed in vivo in the subject by the action of TGs, particularly TG2. Thus, the presence of N′N′ bis(γ-glutamyl)-polyamine dipeptides/isopeptides and/or ε-(γ-glutamyl)-lysine dipeptides/isopeptides, particularly ε-(γ-glutamyl)-lysine dipeptides, in a biological sample from a subject indicates TG activity in the subject.

The enzymes may be capable of cleaving all covalent bonds (including all peptide bonds, intermolecular crosslinks and intramolecular crosslinks) between amino acids in the proteins, except N′N′ bis (γ-glutamyl)-polyamine dipeptide/isopeptide bonds and/or ε-(γ-glutamyl)-lysine dipeptide/isopeptide bonds, particularly ε-(γ-glutamyl)-lysine bonds. Digesting the proteins using enzymes immobilised on beads may release N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides. Thus, the enzymes may digest the proteins to produce a mixture comprising free amino acids and N′N′ bis (γ-glutamyl)-polyamine dipeptides/isopeptides and/or ε-(γ-glutamyl)-lysine dipeptides/isopeptides, particularly free amino acids and ε-(γ-glutamyl)-lysine dipeptides. Thus, in the context of the methods of the present invention, complete protein digestion may comprise cleaving all peptide bonds and crosslinks in the proteins, except N′N′ bis (γ-glutamyl)-polyamine dipeptide/isopeptide bonds and/or ε-(γ-glutamyl)-lysine dipeptide/isopeptide bonds, particularly ε-(γ-glutamyl)-lysine bonds, to produce a mixture of free amino acids and N′N′ bis (γ-glutamyl)-polyamine dipeptides/isopeptides and/or ε-(γ-glutamyl)-lysine dipeptides/isopeptides, particularly a mixture of free amino acids and ε-(γ-glutamyl)-lysine dipeptides. Digestion efficiency provides a measure of the completeness of digestion. 100% digestion efficiency indicates that substantially all of the covalent bonds between amino acids have been cleaved to produce a mixture consisting essentially of free amino acids and N′N′ bis (γ-glutamyl)-polyamine dipeptides/isopeptides and/or ε-(γ-glutamyl)-lysine dipeptides/isopeptides, preferably a mixture consisting essentially of free amino acids and ε-(γ-glutamyl)-lysine dipeptides. In the methods of the invention, the digestion efficiency may be at least 50%, 60%, 70%, 80%, 85%, 90%, 95%, 98% or about 100%, preferably the digestion efficiency is at least 80%.

The enzymes used in the methods of the invention may be selected from endopeptidases, exopeptidases, prolidases, or any combination thereof. The enzymes may comprise a mixture of endopeptidases (i.e., proteolytic enzymes that cleave peptide bonds of nonterminal amino acids, within the polypeptide chain), exopeptidases (i.e., proteolytic enzymes that cleave terminal peptide bonds), and/or prolidases (i.e., enzymes capable of cleaving or hydrolysing the bond between proline and other amino acids, optionally wherein the proline or hydroxyproline residue is located at the C-terminal position). The enzymes may comprise, or consist essentially of, endopeptidases, exopeptidases and prolidases. In some aspects the enzyme may comprise at least an endopeptidase, an exopeptidase and a prolidase.

The enzymes may be selected from proteinase K, pronase, prolidase, leucine aminopeptidase, carboxypeptidase Y, or any combination thereof. In other words, the enzymes may comprise one or more of proteinase K, pronase, prolidase, leucine aminopeptidase, and carboxypeptidase Y. Other enzymes may be present. The enzymes may comprise proteinase K, pronase, prolidase, leucine aminopeptidase, and carboxypeptidase Y; pronase, prolidase, leucine aminopeptidase, and carboxypeptidase Y; proteinase K, prolidase, leucine aminopeptidase, and carboxypeptidase Y; proteinase K, pronase, leucine aminopeptidase, and carboxypeptidase Y; proteinase K, pronase, prolidase, and carboxypeptidase Y; or proteinase K, pronase, prolidase, and leucine aminopeptidase. The enzymes may comprise, or consist essentially of, proteinase K, pronase, prolidase, leucine aminopeptidase and carboxypeptidase Y. The enzymes may comprise, or consist of, a mixture of proteinase K, pronase, prolidase, leucine aminopeptidase and carboxypeptidase Y. In some aspects the enzyme may comprise at least proteinase K, pronase, prolidase, leucine aminopeptidase and carboxypeptidase Y.

The enzymes are immobilised on beads. The enzymes may be covalently linked or conjugated to the beads. Different populations of beads may be provided wherein each population of beads is linked to one type of enzyme. In some aspects proteinase K is immobilised on a first population of beads, pronase is immobilised on a second population of beads, prolidase is immobilised on a third population of beads, leucine aminopeptidase is immobilised on a fourth population of beads and carboxypeptidase Y is immobilised on a fifth population of beads. Thus, in some aspects the enzymes immobilised on beads comprise, or consist essentially of, a mixture of: a first population of beads in which the beads are linked to proteinase K, a second population of beads in which the beads are linked to pronase, a third population of beads in which the beads are linked to prolidase, a fourth population of beads in which the beads are linked to leucine aminopeptidase and a fifth population of beads in which the beads are linked to carboxypeptidase Y. The beads of each population may be the same in all other aspects, except that they are conjugated to different enzymes.

In some aspects digesting the proteins comprises contacting the proteins with the enzymes immobilised on beads. In some aspects digesting the proteins comprises mixing the proteins with the enzymes immobilised on beads and incubating the mixture under conditions suitable for enzyme activity, and thereby digesting the proteins. The exact conditions suitable for enzyme activity may depend on the particular enzyme(s) used for protein digestion. The skilled person would be readily capable of selecting suitable conditions for the selected enzymes. In some aspects, the conditions suitable for enzyme activity may comprise a temperature of 35-38° C. and an incubation time of 16-24 hours. In some aspects, the proteins are digested in the presence of urea. In preferred aspects, the proteins are digested under denaturing conditions (e.g., denaturing urea conditions), which advantageously unfolds the proteins and enhances access of the enzymes. In some aspects, it is advantageous for digesting the proteins to contact the proteins with different enzymes in a particular order. For example, in some preferred aspects digesting the proteins comprises contacting the proteins first with one or more endopeptidase(s) and then contacting the proteins with one or more exopeptidase(s) and/or prolidase(s). This may result in higher digestions efficiencies, for example of at least 80%. Digesting the proteins may comprise multiple (e.g., two, three, four, five, six or more) digestion steps, wherein the proteins are contacted with one or more endopeptidase(s), one or more exopeptidase(s) and one or more prolidase(s). Digesting the proteins may comprise a two-step digestion method wherein the proteins are contacted with an endopeptidase such as proteinase K, and then subsequently contacted with one or more exopeptidases and other enzymes, such as one or more of pronase, prolidase, leucine aminopeptidase and carboxypeptidase Y. Thus, in some aspects, in the methods of the invention, digesting the proteins comprises contacting (e.g., mixing) the proteins with proteinase K immobilised on beads and incubating the mixture under conditions suitable for enzyme activity, and then subsequently contacting the proteins with pronase immobilised on beads, prolidase immobilised on beads, leucine aminopeptidase immobilised on beads and carboxypeptidase Y immobilised on beads, and incubating the mixture under conditions suitable for enzyme activity. Digesting the proteins may comprise contacting the proteins with a first population of beads in which the beads are linked to proteinase K and incubating the mixture under conditions suitable for enzyme activity, and then subsequently adding a second population of beads in which the beads are linked to pronase, a third population of beads in which the beads are linked to prolidase, a fourth population of beads in which the beads are linked to leucine aminopeptidase and a fifth population of beads in which the beads are linked to carboxypeptidase Y and incubating the mixture under conditions suitable for enzyme activity. In some aspects, proteinase K immobilised on beads is mixed with the proteins, the mixture is incubated for about 6-24 hours, preferably about 10-20 hours, even preferably about 16-18 hours, such as about 16, 17 or 18 hours (optionally proteinase K immobilised on beads is added again to the mixture, and incubated for a further about 1-8 hours, preferably about 3-6 hours such as about 4 hours or 6 hours), then pronase, prolidase, leucine aminopeptidase and carboxypeptidase Y, immobilised on beads, are added to the mixture, and the mixture is incubated for a further about 6-36 hours, preferably about 10-24 hours, or even preferably about 14-20 hours such as about 14, 15, 16, 17, 18, 19 or 20 hours, in order to digest the proteins.

The immobilisation of the enzymes on beads is advantageous, as the beads used in the methods of the invention shield the enzymes and prevent self-digestion of the enzymes by the other proteases present in the mixture. This improves the kinetics of digestion of the proteins in the biological sample and dramatically reduces background contamination from the enzymes. The beads typically comprise an outer shielding layer, which layer is configured to reduce or prevent digestion of the enzymes immobilised on the beads by said enzymes. The beads may be silica nanoparticles. The outer shielding layer may be an organosilica layer. The beads may be silica nanoparticles comprising an organosilica outer layer. The enzymes immobilised on the beads may be at least partially embedded in the shielding (e.g., organosilica) layer such that they can still perform their proteolytic function but are themselves protected from being digested by the proteases in the mixture. Such enzymes immobilised on beads are commercially available from Inofea, for example as part of the Enzzen®-Fibrous-Proteins-Digestion kit and are described in more detail in WO2015014888. The proteolytic enzymes are advantageously stabilised, which allows them to be active under advantageous denaturing urea conditions. Additionally, self-digestion (i.e., auto-proteolytic activity) by the enzymes is prevented by the shielding. Thus, the enzyme immobilised on beads used in the method of the invention are both protected and efficient. The present inventors have found that using such enzymes immobilised on beads provides a perfect linearity between protein content in the urine and the occurrence of the dipeptide biomarker, a reproducibility of above 85% and a digestion efficiency of above 90%.

Digesting the proteins using the enzymes immobilised on beads in the methods of the invention releases N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, typically in a background of free amino acids. Typically, the mixture of N′N′ bis(γ-glutamyl)-polyamine dipeptides/isopeptides and/or ε-(γ-glutamyl)-lysine dipeptides/isopeptides, particularly ε-(γ-glutamyl)-lysine dipeptides, and free amino acids is purified and prepared for LC-MS/MS. Suitable procedures for preparing samples for LC-MS/MS are well-known in the art. In some aspects, following enzymatic digestion, the mixture of N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, and free amino acids is purified by solid phase extraction, optionally using a cation exchange cartridge. Typically, this is followed by vacuum-drying the mixture of N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, and free amino acids. The dried mixture of N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, and free amino acids may then be re-suspended in water for subsequent analysis.

The methods of the invention further comprise detecting N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in the biological sample by liquid chromatography-tandem mass spectrometry (LC-MS/MS). The liquid chromatography phase of the LC-MS/MS may be performed via reverse phase chromatography or any other form of liquid chromatography, such as hydrophilic interaction chromatography (HILIC). The LC-MS/MS may use a triple quadrupole mass spectrometer, or any other form of mass spectrometer, such as (but not limited to) a high-resolution accurate mass spectrometer time-of-flight or an orbitrap mass spectrometer. The N′N′ bis (γ-glutamyl)-polyamine dipeptides/isopeptides and/or ε-(γ-glutamyl)-lysine dipeptides/isopeptides, particularly the ε-(γ-glutamyl)-lysine dipeptides, may be detected using LC-MS/MS as having a defined retention time, which may vary in elution, but is calibrated using a stable isotope labelled (SIL) version of the N′N′ bis (γ-glutamyl)-polyamine dipeptide/isopeptide and/or ε-(γ-glutamyl)-lysine dipeptide/isopeptide, particularly a SIL version of the ε-(γ-glutamyl)-lysine dipeptide. The ε-(γ-glutamyl)-lysine dipeptides typically have a retention time equating that of the SIL peptide and have a parent ion mass/charge ratio (m/z) of 276. The peptide can be further detected specifically by monitoring a range of fragment ion masses derived from the parent ion such as m/z 147, 84 and 130. The stable isotope peptide internal standard can be detected with the same retention time and a parent ion m/z of 284, and it can be further detected specifically by monitoring a range of fragment ion masses derived from the parent ion such as m/z 155, 90 and 137. The methods of the invention advantageously allow N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in the biological sample to be accurately and reliably quantified. Thus, the methods of the invention preferably comprise detecting and quantifying N′N′ bis (γ-glutamyl)-polyamine dipeptides/isopeptides and/or ε-(γ-glutamyl)-lysine dipeptides/isopeptides, preferably ε-(γ-glutamyl)-lysine dipeptides, in the biological sample by LC-MS/MS. In such aspects, the method is for detecting and quantifying N′N′ bis (γ-glutamyl)-polyamine dipeptides/isopeptides and/or ε-(γ-glutamyl)-lysine dipeptides/isopeptides, preferably ε-(γ-glutamyl)-lysine dipeptides, in a biological sample.

In some preferred aspects, the method comprises:
a) enriching the proteins present in the biological sample,
b) digesting the proteins using enzymes immobilised on beads, wherein the enzymes comprise proteinase K, pronase, prolidase, leucin aminopeptidase and carboxypeptidase Y, to produce a mixture comprising free amino acids and ε-(γ-glutamyl)lysine dipeptides, and
c) detecting and quantifying the amount of N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in the biological sample by LC-MS/MS, wherein the ε-(γ-glutamyl)-lysine dipeptides are identified as having a retention time equating to the SIL peptide and have a parent ion mass/charge ratio of 276, and further by detecting a range of fragment ion masses derived from the parent ion such as m/z 147, 84 and 130.

In some preferred aspects where the biological sample is a urine sample, the method comprises:
a) enriching the proteins present in the urine sample by precipitating the proteins using trichloroacetic acid,
b) digesting the proteins using enzymes immobilised on beads, wherein the enzymes comprise proteinase K, pronase, prolidase, leucin aminopeptidase and carboxypeptidase Y, to produce a mixture comprising free amino acids and N′N′ bis(γ-glutamyl)-polyamine dipeptides/isopeptides and/or ε-(γ-glutamyl)-lysine dipeptides/isopeptides, preferably a mixture comprising free amino acids and ε-(γ-glutamyl)-lysine dipeptides, and
c) detecting and quantifying the amount of N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in the biological sample by LC-MS/MS, wherein the ε-(γ-glutamyl)-lysine dipeptides are identified as a function of their retention time. Preferably, they have a retention time equating to the SIL peptide and having a parent ion mass/charge ratio of 276, and further by detecting a range of fragment ion masses derived from the parent ion such as m/z 147, 84 and 130.

The above-described methods for detecting crosslinks formed by TGs, such as N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in a biological sample from a subject may also be used to diagnose a disease in the subject. The presence of N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in the biological sample is indicative of TG activity, preferably TG2 activity, in the subject. The amount of N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in the biological sample may be detected and quantified to provide information on the activity of TGs (including Factor XIIIa, TG1, TG2, TG3, TG5, TG4, TG6 and TG7) in vivo in the subject, which may further provide information on disease states within the subject.

Thus, the invention further provides a method for diagnosing a disease, condition or disorder in a subject, the method comprising the steps of:
a) digesting proteins present in a biological sample from the subject using enzymes immobilised on beads, and
b) detecting crosslinks formed by TGs, such as N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in the biological sample by liquid chromatography-tandem mass spectrometry (LC-MS/MS).

Similarly, the invention provides an in vitro method for stratifying the severity of a disease, condition or disorder in a subject, the method comprising the steps of:
a) digesting proteins present in a biological sample from the subject using enzymes immobilised on beads, and
b) detecting crosslinks formed by TGs, such as N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in the biological sample by liquid chromatography-tandem mass spectrometry (LC-MS/MS).

These methods may further comprise any of the features described above in the context of the methods for detecting crosslinks formed by TGs (such as TG2), such as N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, since these diagnostic methods at their core use the above-described methods. Further features of the diagnostic methods are described herein below.

As discussed above, the biological sample is preferably urine or tissue, most preferably urine. The subject may be a mammal, preferably a primate and most preferably a human. Typically, the subject is a human patient having, or suspected of having, a disease. The disease, condition or disorder may be any disease, condition or disorder associated with TG activity, such as TG2 activity. The disease, condition or disorder may be any disease, condition or disorder associated with lowered or reduced TG activity, such as TG2 activity. Preferably, the disease, condition or disorder may be any disease, condition or disorder associated with elevated or increased TG activity, such as TG2 activity. The disease, condition or disorder may be inflammation (e.g., osteoarthritis, idiopathic inflammatory myopathies, rheumatoid arthritis, multiple sclerosis, psoriasis), cancer, fibrosis and fibroproliferative disorders, cardiovascular disease (e.g., coronary heart disease, deep vein thrombosis, vascular calcification, cerebrovascular and peripheral arterial diseases, rheumatic and congenital heart disease), neurodegenerative diseases (e.g., Alzheimer's disease, Parkinson's disease, supranuclear palsy, Huntington's disease and other polyglutamine diseases) or celiac disease. These diseases, conditions or disorders are typically associated with elevated or increased TG (e.g. TG2) activity. The disease, condition or disorder may be cancer. Where the disease, condition or disorder is cancer, it may be colorectal cancer, breast cancer, pancreatic cancer, ovarian cancer, oesophageal squamous cell cancer, glioblastomas, malignant melanomas, renal squamous cell carcinomas, cervical squamous cell carcinomas, hepatocellular carcinomas, cervical intraepithelial neoplasia. Cancers are typically associated with elevated or increased TG (e.g., TG2) activity. The disease, condition or disorder may be fibrosis or a fibroproliferative disorder. Where the disease, condition or disorder is fibrosis or a fibroproliferative disorder, it may be, without limitation, chronic kidney disease (including e.g., post-transplant renal fibrosis or chronic allograft nephropathy (CAN)), progressive kidney disease, pulmonary fibroses, systemic sclerosis, liver cirrhosis, cardiovascular disease, idiopathic hypertrophic cardiomyopathy, renal fibrosis, primary glomerulonephritis, liver cirrhosis, chronic allograft injury (CAI), or diabetic nephropathy. In some aspects, the disease, condition or disorder is post-transplant renal fibrosis (including e.g., CAI, or CAN). Fibrosis and fibroproliferative disorders are typically associated with elevated or increased TG, such as TG2, activity.

In some aspects the method further comprises quantifying and/or determining the amount/number of crosslinks formed by TGs, such as N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in the biological sample. The method may further comprise comparing the amount of N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in the biological sample to a control value. The control value may be the amount of N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, detected in a biological sample from a subject not having the disease, disorder or condition, preferably a healthy subject. The control value may be the average amount of N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, detected in biological samples from a number of subjects not having the disease, disorder or condition, preferably a number of healthy subjects. The control value may be determined using the methods of the invention. The control value may vary depending on the type of biological sample used in the methods of the invention. In some aspects, no N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, are detected using the methods of the invention in biological samples (such as urine samples) from healthy human subjects (i.e., subjects not having the disease, disorder or condition). Thus, in some aspects the control value may be zero. In some other aspects, in a healthy human subject, the amount of N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in a biological sample, as determined using the methods of the invention may be X±Y ng crosslink/mg protein; or ng crosslink/unit creatine; or ng crosslink/ml biological sample (such as urine), preferably ng crosslink/ml biological sample (such as urine). The most suitable units typically depend on the type of biological sample being used. For example, for urine samples based on a 24-hour urine collection the units are typically ng crosslink/ml urine. Thus, in some aspects, the control value may be X±Y. The skilled person would readily understand that the control value does not need to be determined at the same time as a test value is determined. Typically, the control value is established in advance of testing, for example by determining the amount of N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in a selected biological sample type from a cohort of healthy subjects.

In some aspects, no N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, would be detected using the methods of the invention in a biological sample from a healthy subject and in such instances, comparing to a control value is not required. In such aspects, detecting essentially no (e.g., zero) N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in the biological sample indicates that the subject has normal (i.e., healthy) TG (e.g., TG2) activity and/or that the subject does not have the disease, disorder or condition. Whereas, detecting N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in the biological sample (i.e., a non-zero amount of ε-(γ-glutamyl)-lysine dipeptides) using the methods of the invention indicates that the subject has the disease, disorder or condition. In some other aspects, an amount of N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in the biological sample that is equal to, or within ±Z of the control value indicates that the subject has normal (i.e., healthy) TG (e.g. TG2) activity and/or the subject does not have the disease, disorder or condition. In some aspects, for example where the disease is associated with reduced TG (e.g., TG2) activity, an amount of N′N′ bis (γ-glutamyl)-polyamine dipeptides/isopeptides and/or ε-(γ-glutamyl)-lysine dipeptides/isopeptides, preferably ε-(γ-glutamyl)-lysine dipeptides, in the biological sample that is lower than the control value (e.g., significantly lower), indicates that the subject has the disease, disorder or condition. An amount of N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in the biological sample that is less than 0.95 times, 0.9 times, 0.8 times, 0.7 times, 0.6 times, 0.5 times, 0.4 times, 0.2 times, or less than 0.1 times the control value, preferably less than 0.8 times the control value, most preferably less than 0.5 times the control value, may indicate that the subject has the disease, disorder or condition. In some aspects, at least a 10%, 15%, 20%, 25%, 30%, 40%, 50%, 60%, 70%, or at least an 80% decrease, preferably at least a 10% decrease, most preferably at least a 20% decrease, in the amount of N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in the biological sample as compared to the control value may indicate that the subject has the disease, disorder or condition. Typically, the disease, condition or disorder is more likely to be associated with elevated TG (e.g., TG2) activity. Thus, in preferred aspects, for example where the disease is associated with elevated TG (e.g., TG2) activity, an amount of N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in the biological sample that is higher than the control value (e.g., significantly higher), indicates that the subject has the disease, disorder or condition. An amount of ε-(γ-glutamyl)-lysine dipeptides in the biological sample that is at least 1.1 times, 1.2 times, 1.3 times, 1.4 times, 1.5 times, 2 times, 2.5 times, 3 times, 4 times, 5 times, 10 times, or at least 20 times the control value, preferably at least 1.2 times the control value, most preferably at least 1.5 times the control value, may indicate that the subject has the disease, disorder or condition. In some aspects, at least a 2%, 5%, 10%, 15%, 20%, 25%, 30%, 40%, 50%, 60%, 70%, 80%, 90%, 100%, 150%, or at least an 200% increase, preferably at least a 10% increase, most preferably at least a 20% increase, in the amount of N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in the biological sample as compared to the control value may indicate that the subject has the disease, disorder or condition.

The methods according to the invention allow for the selective detection and/or quantification of N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, even at low concentrations in a biological sample. In one embodiment, the Lower Limit of Quantification (LLOQ) is as low as about 0.1 ng crosslink/mL of biological sample (such as urine). The Upper Limit of Quantification (ULOQ) of crosslinks according to the method can be at least as high as about 50 ng crosslink/mL of biological sample (such as urine). Suitably, the methods can (accurately) detect and/or quantify crosslinks in the biological sample at 1) a LLOQ of, for example, about 0.1 ng crosslink/mL of biological sample (such as urine) or more, and/or 2) a ULOQ of, for example, about 50 ng crosslink/mL of biological sample (such as urine) or less. In other words, the methods according to the present invention can (accurately) detect and/or quantify crosslinks in an amount of from about 0.1 ng to about 50 ng crosslink/mL of biological sample (such as urine). In another example, the methods of the invention can (accurately) detect and/or quantify crosslinks in the biological sample in the range from about 0.1 to about 40 ng crosslink/mL biological sample (such as urine), preferably from about 0.1 to about 20 ng crosslink/mL biological sample (such as urine), most preferably from about 0.1 to about 15 ng crosslink/mL biological sample (such as urine), from about 0.1 to about 10 ng crosslink/mL biological sample (such as urine), such as about 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5 or 10 ng crosslink/mL biological sample (such as urine).

In some exemplary, but not limiting, aspects, in a human patient having chronic kidney disease such as post-transplant renal fibrosis, CAI, or CAN, the amount of N′N′ bis(γ-glutamyl)-polyamine dipeptides/isopeptides and/or ε-(γ-glutamyl)-lysine dipeptides/isopeptides, particularly ε-(γ-glutamyl)-lysine dipeptides, in a urine sample, as determined using the methods of the invention may be at least 0.1 ng crosslink/mL urine in line with the LLOQ (lower limit of quantification of the LC-MS/MS measurement), such as from about 0.1 to about 50 ng crosslink/mL urine, from about 0.1 to about 40 ng crosslink/mL urine, from about 0.1 to about 20 ng crosslink/mL urine, from about 0.1 to about 10 ng crosslink/mL urine or yet from about 0.1 to about 5 ng crosslink/mL. For urine samples, the reported concentration of ε-(γ-glutamyl) lysine may be normalized to mg protein measured in the bicinchoninic acid (BCA) method and expressed as ng ε-(γ-glutamyl) lysine equivalent per mg of protein. In such aspects, the amount of N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in a urine sample from a control healthy subject, as determined using the methods of the invention, is typically 0 ng crosslink/mL urine. This is typically because the crosslink and protein values in a urine sample from a healthy subject are lower than the sensitivity threshold of the methods of the invention.

The amount of N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in the biological sample can also be used to stratify the severity or prognosis of the disease. In some aspects, for example where the disease is associated with reduced TG activity, such as TG2 activity, the lower the amount of N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in the biological sample the more severe the disease and/or the poorer the prognosis of the disease. In preferred aspects, for example where the disease is associated with elevated TG activity, such as TG2 activity, the higher the amount of N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in the biological sample the more severe the disease and/or the poorer the prognosis of the disease. The disease may be categorised as ‘severe’ or as having a ‘poor prognosis’ based on a threshold value. In some aspects, for example where the disease is associated with reduced TG activity, such as TG2 activity, an amount of N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in the biological sample that is lower than the threshold value indicates that the disease can be categorised as severe and/or having a poor prognosis. In some preferred aspects, for example where the disease is associated with elevated TG activity, such as TG2 activity, an amount of N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in the biological sample that is the same or higher than the threshold value indicates that the disease can be categorised as severe and/or having a poor prognosis. In such aspects, an amount of N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in the biological sample that is lower than the threshold value indicates that the disease can be categorised as normal and/or having a normal prognosis. The threshold value may be the amount of N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, detected in a biological sample from a subject having the disease, disorder or condition that is known to be severe or have a poor prognosis, for example as determined using other assays or clinical factors. The threshold value may be the average amount of N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, detected in biological samples from a number of subjects having the disease, disorder or condition that is known to be severe or have a poor prognosis. The threshold value may be determined using the methods of the invention. In some aspects, for example where the disease is associated with reduced TG activity, such as TG2 activity, the threshold value may be 0.6 times, 0.5 times, 0.4 times, 0.2 times, or less than 0.1 times the control value, preferably 0.5 times the control value, most preferably 0.2 times the control value. In some aspects, for example where the disease is associated with elevated TG activity, such as TG2 activity, the threshold value may be 1.5 times, 2 times, 2.5 times, 3 times, 4 times, 5 times, 10 times, or at least 20 times the control value, preferably at least 2 times the control value, most preferably at least 5 times the control value. In some aspects, for example where the disease is associated with reduced TG activity, such as TG2 activity, the threshold value may be at least 10%, 15%, 20%, 25%, 30%, 40%, 50%, 60%, 70%, 80%, 90% or at least 100% lower, preferably at least 20% lower, most preferably at least a 50% lower than the control value. In some preferred aspects, for example where the disease is associated with elevated TG activity, such as TG2 activity, the threshold value may be at least 10%, 15%, 20%, 25%, 30%, 40%, 50%, 60%, 70%, 80%, 90%, 100%, 150%, or at least 200% higher, preferably at least 20% higher, most preferably at least 50% higher, than the control value. For example, in a human patient having a severe form of a disease, condition or disorder as defined herein, the amount of N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in a biological sample, as determined using the methods of the invention, may be Q±R ng crosslink/mg protein; or ng crosslink/unit creatine; or ng crosslink/ml biological sample (such as urine), preferably ng crosslink/ml biological sample (such as urine). The most suitable units typically depend on the type of biological sample being used. For example, for urine samples based on a 24-hour urine collection the units are typically ng crosslink/ml urine. Thus, in some aspects, the threshold value may be Q±R.

The methods of the invention can also be used for monitoring the progression of a disease, disorder or condition in a subject, i.e., whether the disease, disorder or condition is improving or getting worse in the subject over time. Thus, the invention provides an in vitro method for monitoring the progression of a disease, disorder or condition in a subject, the method comprising the steps of:
a) digesting proteins present in a biological sample from the subject using enzymes immobilised on beads; and
b) detecting crosslinks formed by TGs, such as N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in the biological sample by liquid chromatography-tandem mass spectrometry (LC-MS/MS); and
c) comparing the amount of crosslinks formed by TGs, such as N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in the biological sample with the amount of crosslinks formed by TGs, such as N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in a previous sample from the patient or with a control value.

Preferably, as described above, the disease is any disease associated with reduced or preferably elevated TG activity, such as TG2 activity. Preferably, the disease is fibrosis, a fibrotic disease or disorder or a fibrosis-related disease or disorder. Such diseases or disorders can be (without any limitations) chronic kidney disease (including post-transplant renal fibrosis and chronic allograft nephropathy (CAN)), progressive kidney disease, pulmonary fibroses, systemic sclerosis, liver cirrhosis, cardiovascular disease, idiopathic hypertrophic cardiomyopathy, renal fibrosis, primary glomerulonephritis, liver cirrhosis, or chronic allograft injury (CAI).

These diseases or disorders are typically all associated with an elevated or increased TG2 activity. These methods may further comprise any of the features described above in the context of the methods for detecting crosslinks formed by TGs, such as N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, or the methods for diagnosis.

The method may further comprise determining the amount of N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in the biological samples. The amount of N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in the previous biological sample from the subject may also be determined using the methods of the invention. Typically, the first biological sample was obtained from the subject at least 1 month, 2 months, 3 months, 6 months, 9 months, 12 months, 18 months, 2 years, 5 years, or at least 10 years, preferably at least 6 months, most preferably at least 12 months, before the second biological sample was obtained from the subject. Thus, changes in the amount/number of dipeptides in the biological sample from the subject can be monitored, which allows the TG, such as TG2, activity to be monitored, which in turn allows the disease to be monitored.

In some aspects, for example where the disease is associated with reduced TG activity, such TG2 activity, an increase in the amount of N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in the second biological sample as compared to the amount of N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in the first biological sample indicates that the disease is improving. In some aspects, for example where the disease is associated with reduced TG activity, such as TG2 activity, a decrease in the amount of N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in the second biological sample as compared to the amount of N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in the first biological sample indicates that the disease is worsening. In preferred aspects, for example where the disease is associated with elevated TG activity, such TG2 activity, a decrease in the amount of N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in the second biological sample as compared to the amount of N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in the first biological sample indicates that the disease is improving. In preferred aspects, for example where the disease is associated with elevated TG activity, such as TG2 activity, an increase in the amount of N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in the second biological sample as compared to the amount of N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in the first biological sample indicates that the disease is worsening. In each case, the increase or decrease in the amount of N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, is preferably a significant increase or decrease. In some aspects, an amount of N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in the second biological sample that is at least 5%, 10%, 15%, 20%, 25%, 30%, 40%, 50%, 60%, 70%, 80%, 90%, 100%, 150%, or at least 200% higher, preferably at least 10% higher, most preferably at least 20% higher, than the amount of N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in the first biological sample, indicates that the disease is worsening.

The method may further comprise obtaining one or more further biological sample(s) at time intervals of 1 month, 2 months, 3 months, 6 months, 9 months, 12 months, 18 months, 2 years, 5 years, or 10 years, preferably at time intervals of 6 months, most preferably at time intervals of 12 months, and performing the method to monitor the progression of the disease over a number of years. The method may further comprise comparing the amount of N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in the one or more biological samples from the subject with the control value, as defined herein. An increase in the difference from the control value over time indicates that the disease, disorder or condition is worsening in the subject. A decrease in the difference from the control value (i.e., the amount getting closer towards the control value) over time indicates that the disease, disorder or condition is improving in the subject.

The methods of the invention allow for detecting crosslinks formed by TGs, such as N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in a biological sample from a subject, which provides an indication of the TG activity in the subject and may be indicative of a disease state. Similarly, then monitoring the levels of crosslinks formed by TGs, such as N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in a biological sample from a subject may also be used to infer whether a treatment is effective or whether an agent targeting TG is effective. The present invention therefore provides a method for determining a subject's response to a treatment, the method comprising the steps of:
a) digesting proteins present in a biological sample from the subject using enzymes immobilised on beads; and
b) detecting crosslinks formed by TGs, such as N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in the biological sample by liquid chromatography-tandem mass spectrometry (LC-MS/MS); and
c) comparing the amount of crosslinks formed by TGs, such as N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in the biological sample with the amount of crosslinks formed by TGs, such as N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in a biological sample from the patient prior to treatment, or at an earlier stage in treatment, or with a control value.

Preferably, as described above, the subject is a human patient. The subject may preferably be a human patient having a disease associated with reduced or preferably elevated TG activity, such as TG2 activity. Preferably, the disease is fibrosis, a fibrotic disease or disorder or a fibrosis-related disease or disorder. Such diseases or disorders can be (without any limitations) chronic kidney disease (including post-transplant renal fibrosis and chronic allograft nephropathy), progressive kidney disease, pulmonary fibroses, systemic sclerosis, liver cirrhosis, cardiovascular disease, idiopathic hypertrophic cardiomyopathy, renal fibrosis, primary glomerulonephritis, liver cirrhosis, or chronic allograft injury (CAI). The treatment may therefore be any treatment for a disease associated with reduced or preferably elevated TG activity, such as TG2 activity. In some aspects, the treatment is for fibrosis, a fibrotic disease or disorder or a fibrosis-related disease or disorder. Such diseases or disorders can be (without any limitations) chronic kidney disease (including post-transplant renal fibrosis and chronic allograft nephropathy), progressive kidney disease, pulmonary fibroses, systemic sclerosis, liver cirrhosis, cardiovascular disease, idiopathic hypertrophic cardiomyopathy, renal fibrosis, primary glomerulonephritis, liver cirrhosis, or chronic allograft injury. All of these diseases or disorders are typically associated with elevated or increased TG2 activity.

In some aspects, for example where the disease is associated with reduced TG activity, such as TG2 activity, the method may comprise comparing (i) the amount of N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in the biological sample with (ii) the amount of N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in a biological sample from a different subject having the disease who has not been administered the treatment; wherein a higher amount of N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in (i) as compared to (ii) indicates that the treatment is effective. In some aspects, for example where the disease is associated with reduced TG activity, such as TG2 activity, the method may comprise comparing (i) the amount of N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in the biological sample with (ii) the amount of N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in a biological sample taken from the subject prior to treatment, or at an earlier stage in treatment; wherein a higher amount of N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in (i) as compared to (ii) indicates that the treatment is effective.

In some preferred aspects, for example where the disease is associated with elevated TG activity, such as TG2 activity, the method may comprise comparing (i) the amount of N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in the biological sample with (ii) the amount of N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in a biological sample from a different subject having the disease who has not been administered the treatment; wherein a lower amount of N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in (i) as compared to (ii) indicates that the treatment is effective. In some preferred aspects, for example where the disease is associated with elevated TG activity, such as TG2 activity, the method may comprise comparing (i) the amount of N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in the biological sample with (ii) the amount of N′N′ bis(γ-glutamyl)-polyamine dipeptides/isopeptides and/or ε-(γ-glutamyl)-lysine dipeptides/isopeptides, particularly ε-(γ-glutamyl)-lysine dipeptides, in a biological sample taken from the subject prior to treatment, or at an earlier stage in treatment; wherein a lower amount of N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides in (i) as compared to (ii) indicates that the treatment is effective. In some aspects, for example where the disease is associated with elevated TG activity, such as TG2 activity, the method may comprise comparing (i) the amount of N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in the biological sample with (ii) the amount of N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in a biological sample taken from the subject prior to treatment, or at an earlier stage in treatment; wherein no significant change in the amount of N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in (i) as compared to (ii) indicates that the treatment is effective.

In some aspects, immediately following administration of the treatment an increased amount of N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in the biological sample may be observed as partly crosslinked tissue turns over more rapidly, but over several weeks the crosslink is slowly excised as the tissue turns over and the extracellular matrix containing the crosslink is digested and the amount of N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in the biological sample typically decreases to very lower levels. Thus, in some aspects, the method may comprise comparing (i) the amount of N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in the biological sample with (ii) the amount of N′N′ bis(γ-glutamyl)-polyamine dipeptides/isopeptides and/or ε-(γ-glutamyl)-lysine dipeptides/isopeptides, particularly ε-(γ-glutamyl)-lysine dipeptides, in a biological sample taken from the subject prior to treatment, or at an earlier stage in treatment; wherein an initial increase in the amount of N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in (i) as compared to (ii) (e.g., in weeks 1, 2, and/or 3 following administration of the treatment) followed by a decrease in the amount of N′N′ bis(γ-glutamyl)-polyamine dipeptides/isopeptides and/or ε-(γ-glutamyl)-lysine dipeptides/isopeptides, particularly ε-(γ-glutamyl)-lysine dipeptides, in (i) as compared to (ii) (e.g., at least 3, 4, 5, 6, 7, 8, or more after administration of the treatment) indicates that the treatment is effective.

The method may comprise obtaining a biological sample from the subject prior to treatment. The method may comprise administering the treatment to the subject. The method may comprise obtaining a biological sample from the subject after administration of the treatment to the subject. The treatment may be a TG antagonist or a TG inhibitor, such as an anti-TG antibody (e.g. anti-TG2 antibody) or a small molecule.

Similarly, the invention also provides a method for determining the effect of an agent on TG activity, such as TG2 activity, the method comprising the steps of:
a) digesting proteins present in a biological sample, from a subject administered the agent, using enzymes immobilised on beads, and
b) detecting crosslinks formed by TGs, such as N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in the biological sample by liquid chromatography-tandem mass spectrometry (LC-MS/MS).

The method may further comprise comparing (i) the amount of N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in the biological sample with (ii) the amount of N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in a biological sample from a different subject who has not been administered the agent; wherein a difference in the amount of N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in (i) as compared to (ii) indicates that the agent is having an effect. The method may further comprise comparing (i) the amount of N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in the biological sample with (ii) the amount of N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in a biological sample taken from the subject prior to administration of the agent; wherein a difference in the amount of N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in (i) as compared to (ii) indicates that the agent is having an effect.

The method may comprise obtaining a biological sample from the subject prior to treatment. The method may comprise administering the treatment to the subject. The method may comprise obtaining a biological sample from the subject after administration of the treatment to the subject. The treatment may be a TG antagonist or a TG inhibitor such as an anti-TG antibody (e.g. anti-TG2 antibody) or a small molecule.

A difference in the amount of N′N′ bis (γ-glutamyl)-polyamine dipeptides/isopeptides and/or ε-(γ-glutamyl)-lysine dipeptides/isopeptides, particularly ε-(γ-glutamyl)-lysine dipeptides, in (i) as compared to (ii) may indicate target engagement by the agent. Where the difference is an increase, this typically indicates that the agent is a TG activator (i.e. an activator of one or more of Factor XIIIa, TG1, TG2, TG3, TG5, TG4, TG6 or TG7). For example, the agent may be a TG or TG2 agonist, and/or the agent may increase the expression or activity of TG, such as TG2. Where the difference is a decrease, this indicates that the agent is a TG inhibitor (i.e. an inhibitor to one or more of Factor XIIIa, TG1, TG2, TG3, TG5, TG4, TG6 or TG7). For example, the agent may inhibit substrate binding by TG (such as TG2) and/or inhibit or decrease the expression or activity of TG (such as TG2). The agent may bind to the TG, such as TG2, or a substrate thereof, and prevent or reduce binding of the TG, such as TG2, to its substrate. The agent may be a polynucleotide, a polypeptide, an antibody or a small molecule. The agent may be an anti-TG antibody, such as an anti-TG2 antibody. Where sample (i) is taken within 1, 2, 3, or 4 weeks of administration of an agent that is a TG inhibitor, such as a TG2 inhibitor, an increase may be observed due to rapid turnover of crosslinked tissue, but typically after 3, 4, 5, 6, 7, 8 or more months following administration of the agent a decrease will be observed. The method may comprise obtaining a biological sample from the subject prior to administration of the agent. The method may comprise administering the agent to the subject. The method may comprise obtaining a biological sample from the subject after administration of the agent to the subject.

The term TG inhibitor, as used herein, is intended to refer to a molecule that binds and inhibit TG (i.e., an inhibitor to one or more of Factor XIIIa, TG1, TG2, TG3, TG5, TG4, TG6 or TG7). The term “anti-TG antibody”, as used herein, is intended to refer to an antibody molecule which binds to one or more TG (i.e., to one or more of Factor XIIIa, TG1, TG2, TG3, TG5, TG4, TG6 or TG7). The term “anti-TG2 antibody”, as used herein, is intended to be an antibody molecule which binds TG2. Examples of such antibodies are described in WO2013175229. Without any limitation, an anti-TG2 antibody that can be used according to the present invention:
a) comprises 6 CDRs selected from the group consisting of:

(i)
(LCDR1; SEQ ID NO. 1)
KASQDINSYLT;
(LCDR2; SEQ ID NO. 2)
LVNRLVD;
(LCDR3; SEQ ID NO. 3)
LQYDDFPYT;
(HCDR1; SEQ ID NO. 4)
THAMS;
(HCDR2; SEQ ID NO. 5)
TISSGGRSTYYPDSVKG;
and
(HCDR3; SEQ ID NO. 6)
LISTY;
or
(ii)
(LCDR1; SEQ ID NO. 1)
KASQDINSYLT;
(LCDR2; SEQ ID NO. 7);
LTNRLMD
(LCDR3; SEQ ID NO. 8)
LQYVDFPYT;
(HCDR1; SEQ ID NO. 9)
SSAMS;
(HCDR2; SEQ ID NO. 5)
TISSGGRSTYYPDSVKG;
and
(HCDR3; SEQ ID NO. 10)
LISPY;
or
(iii)
(LCDR1; SEQ ID NO. 1)
KASQDINSYLT;
(LCDR2; SEQ ID NO. 11)
RTNRLFD;
(LCDR3; SEQ ID NO. 3)
LQYDDFPYT;
(HCDR1)
SSAMS;
(HCDR2; SEQ ID NO. 9)
TISVGGGKTYYPDSVKG;
and
(HCDR3; SEQ ID NO. 12)
LISLY.

b) comprises a light chain variable domain having the sequence as defined in any one of SEQ ID NO: 13 to SEQ ID NO: 27 and a heavy chain variable domain having the sequence as defined in any one of SEQ ID NO: 28 to SEQ ID NO: 40,
c) comprises a light chain variable domain having at least 80% identity or similarity, preferably 90% identity or similarity to the sequence as defined in any one of SEQ ID NO: 13 to SEQ ID NO: 27 and a heavy chain variable domain having at least 80% identity or similarity, preferably 90% identity or similarity to the sequence as defined in any one of SEQ ID NO: 28 to SEQ ID NO: 40, or
d) competes for binding to an epitope comprising or consisting of amino acids 304 to 326 of human TG2 (see SEQ ID NO:41) or part of this region, with an antibody as defined in a), b) or c) above.

TABLE A
Anti-TG2 and TG2 amino acid sequences

| SEQ ID | Amino acid sequence |
|---|---|
| 1 | KASQDINSYLT |
| 2 | LVNRLVD |
| 3 | LQYDDFPYT |
| 4 | THAMS |
| 5 | TISSGGRSTYYPDSVKG |
| 6 | LISTY |
| 7 | LTNRLMD |
| 8 | LQYVDFPYT |
| 9 | SSAMS |
| 10 | LISPY |
| 11 | RTNRLFD |
| 12 | LISLY |
| 13 | DIQMTQTPSSMYASLGERVTITCKASQDINSYLTWFQQKPGKSPKTLIYR |
| | TNRLFDGVPSRFSGSGSGQDFFLTISSLEYEDMGIYYCLQYDDFPYTFG |
| | GGTKLEIK |
| 14 | DIKMTQSPSSMYASLGERVTITCKASQDINSYLTWFQQKPGKSPKTLIYR |
| | TNRLFDGVPSRFSGSGSGQDFFLTISSLEYEDMGIYYCLQYDDFPYTFG |
| | GGTKLEIK |
| 15 | EIVLTQSPSSMYASLGERVTITCKASQDINSYLTWFQQKPGKSPKTLIYRT |
| | NRLFDGVPSRFSGSGSGQDFFLTISSLEYEDMGIYYCLQYDDFPYTFGG |
| | GTKLEIK |
| 16 | DIQMTQSPSSMYASLGERVTITCKASQDINSYLTWFQQKPGKSPKTLIYR |
| | TNRLFDGVPSRFSGSGSGQDFFLTISSLEYEDMGIYYCLQYDDFPYTFG |
| | GGTKLEIK |
| 17 | EIVLTQSPSSLSASVGDRVTITCKASQDINSYLTWYQQKPGKAPKLLIYRT |
| | NRLFDGVPSRFSGSGSGTDFFFTISSLQPEDFGTYYCLQYDDFPYTFGG |
| | GTKLEIK |
| 18 | DIQMTQSPSSLSASVGDRVTITCKASQDINSYLTWFQQKPGKAPKSLIYR |
| | TNRLFDGVPSRFSGSGSGTDFFLTISSLQPEDFATYYCLQYDDFPYTFG |
| | QGTKVEIK |
| 19 | AIKMTQSPSSMYASLGERVIITCKASQDINSYLTWFQQKPGKSPKTLIYLT |
| | NRLMDGVPSRFSGSGSGQEFLLTISGLEHEDMGIYYCLQYVDFPYTFGG |
| | GTKLEIK |
| 20 | DIQMTQSPSSLSASVGDRVTITCKASQDINSYLTWFQQKPGKAPKSLIYL |
| | TNRLMDGVPSRFSGSGSGTDFFLTISSLQPEDFATYYCLQYVDFPYTFG |
| | QGTKVEIK |
| 21 | DIKMTQSPSSLSASVGDRVTITCKASQDINSYLTWFQQKPGKAPKTLIYLT |
| | NRLMDGVPSRFSGSGSGQEFLLTISSLQPEDFATYYCLQYVDFPYTFGQ |
| | GTKVEIK |
| 22 | DITMTQSPSSIYASLGERVTITCKASQDINSYLTWFQQKPGKSPKILIYLVN |
| | RLVDGVPSRFSGSGSGQDYALTISSLEYEDMGIYYCLQYDDFPYTFGGG |
| | TKLEIK |
| 23 | DIQMTQSPSSLSASVGDRVTITCKASQDINSYLTWFQQKPGKAPKSLIYL |
| | VNRLVDGVPSRFSGSGSGTDFFLTISSLQPEDFATYYCLQYDDFPYTFG |
| | QGTKVEIK |
| 24 | DITMTQSPSSLSASVGDRVTITCKASQDINSYLTWFQQKPGKAPKILIYLV |
| | NRLVDGVPSRFSGSGSGQDYALTISSLQPEDFATYYCLQYDDFPYTFGQ |
| | GTKVEIK |
| 25 | AIKMTQTPSSVSAAVGGTVTISCKASQDINSYLTWFQQKPGQPPKTLIYL |
| | TNRLMDGVPSRFSGSGSGQEFLLTISGLECDDAATYYCLQYVDFPYTFG |
| | GGTKVVVK |
| 26 | DVVNTQTPLTLSVTFGQPASISCKSSQSLLYDNGKTYLHWLFQRPGQSP |
| | RRLIYLVSKLDSGVPDRFTGSGSGTDFTLKISRVEAEDLGVYYCVQGTHF |
| | PYTFGGGTKLEIK |
| 27 | QIVLTQSPAIMSASPGEKVTMTCSASSSVSYMYWYQQKPGSSPRLLIYD |
| | TSNLASGVPVRFSGSGSGTSYSLTISRMEAEDAATFYCQQWSSSPLTFG |
| | AGTKLELK |
| 28 | EVQLEESGGGLVKPGGSLKLSCAASGFTLSSSAMSWVRQTPDRRLEWV |
| | ATISVGGGKTYYPDSVKGRFTISRDNAKNTLYLQMNSLRSEDTAMYYCA |
| | KLISLYWGQGTTLTVSS |
| 29 | EVQLVESGGGLVKPGGSLKLSCAASGFTLSSSAMSWVRQTPDRRLEWV |
| | ATISVGGGKTYYPDSVKGRFTISRDNAKNTLYLQMNSLRSEDTAMYYCA |
| | KLISLYWGQGTTLTVSS |
| 30 | EVQLQESGGGLVKPGGSLKLSCAASGFTLSSSAMSWVRQTPDRRLEW |
| | VATISVGGGKTYYPDSVKGRFTISRDNAKNTLYLQMNSLRSEDTAMYYC |
| | AKLISLYWGQGTTLTVSS |
| 31 | EVQLLESGGGLVQPGGSLRLSCAASGFTFSSSAMSWVRQAPGKGLEW |
| | VSTISVGGGKTYYPDSVKGRFTISRDNSKNTLYLQMNSLRAEDTAVYYC |
| | AKLISLYWGQGTLVTVSS |
| 32 | AVQLVESGGGLVKPGGSLKLSCAASGIIFSSSAMSWVRQTPEKRLEWVA |
| | TISSGGRSTYYPDSVKGRFTVSRDSAKNTLYLQMDSLRSEDTAIYYCAKL |
| | ISPYWGQGTTLTVSS |
| 33 | EVQLLESGGGLVQPGGSLRLSCAASGFTFSSSAMSWVRQAPGKGLEW |
| | VSTISSGGRSTYYPDSVKGRFTISRDNSKNTLYLQMNSLRAEDTAVYYCA |
| | KLISPYWGQGTLVTVSS |
| 34 | EVQLLESGGGLVQPGGSLRLSCAASGIIFSSSAMSWVRQAPGKGLEWV |
| | ATISSGGRSTYYPDSVKGRFTVSRDSSKNTLYLQMNSLRAEDTAVYYCA |
| | KLISPYWGQGTLVTVSS |
| 35 | EVQLVESGGGLVKPGGSLKLSCAASGFTLSTHAMSWVRQTPEKRLEWV |
| | ATISSGGRSTYYPDSVKGRFTISRDNVKNTLYLQLSSLRSEDTAVYFCAR |
| | LISTYWGQGTTLTVSS |
| 36 | EVQLLESGGGLVQPGGSLRLSCAASGFTFSTHAMSWVRQAPGKGLEW |
| | VSTISSGGRSTYYPDSVKGRFTISRDNSKNTLYLQMNSLRAEDTAVYYCA |
| | KLISTYWGQGTLVTVSS |
| 37 | EVQLLESGGGLVQPGGSLRLSCAASGFTLSTHAMSWVRQAPGKGLEW |
| | VATISSGGRSTYYPDSVKGRFTISRDNSKNTLYLQMNSLRAEDTAVYFCA |
| | RLISTYWGQGTLVTVSS |
| 38 | EVQLLESGGRLVTPGGSLTLTCAASGIIFSSSAMSWVRQAPGKGLEWVA |
| | TISSGGRSTYYPDSVKGRFTVSRDSSKNTLYLQMNSLRAEDTAVYYCAK |
| | LISPYWGQGTLVTVSS |
| 39 | QIQLVQSGPELKKPGETVKISCKASGYTFTTYGNTWVKQAPGKGLKWM |
| | GWINTSSGVPTYADDFKGRFAFSLETSASTAYLQINNLKSEDTATYFCAR |
| | PEVAYWGQGTLVTVSA |
| 40 | QVTLKESGPGILQPSQTLSLTCSFSGFSLSTSGMGVSWIRQPSGKGLEW |
| | LAHIYWDDDKRYNPSLKSRLTISKDTSSNQVFLKITSVDTADTATYYCARS |
| | GTTAPFAYWGQGTLVTVSA |
| 41 | AEELVLERCDLELETNGRDHHTADLCREKLVVRRGQPFWLTLHFEGRNY |
| | EASVDSLTFSVVTGPAPSQEAGTKARFPLRDAVEEGDWTATVVDQQDC |
| | TLSLQLTTPANAPIGLYRLSLEASTGYQGSSFVLGHFILLFNAWCPADAVY |
| | LDSEEERQEYVLTQQGFIYQGSAKFIKNIPWNFGQFEDGILDICLILLDVN |
| | PKFLKNAGRDCSRRSSPVYVGRVVSGMVNCNDDQGVLLGRWDNNYGD |
| | GVSPMSWIGSVDILRRWKNHGCQRVKYGQCWVFAAVACTVLRCLGIPT |
| | RVVTNYNSAHDQNSNLLIEYFRNEFGEIQGDKSEMIWNFHCWVESWMT |
| | RPDLQPGYEGWQALDPTPQEKSEGTYCCGPVPVRAIKEGDLSTKYDAP |
| | FVFAEVNADVVDWIQQDDGSVHKSINRSLIVGLKISTKSVGRDEREDITHT |
| | YKYPEGSSEEREAFTRANHLNKLAEKEETGMAMRIRVGQSMNMGSDFD |
| | VFAHITNNTAEEYVCRLLLCARTVSYNGILGPECGTKYLLNLNLEPFSEKS |
| | VPLCILYEKYRDCLTESNLIKVRALLVEPVINSYLLAERDLYLENPEIKIRILG |
| | EPKQKRKLVAEVSLQNPLPVALEGCTFTVEGAGLTEEQKTVEIPDPVEAG |
| | EEVKVRMDLLPLHMGLHKLVVNFESDKLKAVKGFRNVIIGPA (human |
| | TG2 Amino Acid sequence) |
| 42 | MAEELNLERCDLEIQANGRDHHTADLCQEKLVLRRGQRFRLTLYFEGRG |
| | YEASVDRLTFGAVTGPDPSEEAGTKARFSLSDDVEEGSWSASVLDQQD |
| | NVLSLQLCTPANAPVGQYRLSLETSTGYQGSSFMLGHFILLFNAWCPAD |
| | DVYLDSEAERREYVLTQQGFIYQGSVKFIKSVPWNFGQFEDGILDACLML |
| | LDVNPKFLKDRSRDCSRRSSPIYVGRVVSGMVNCNDDQGVLLGRWDN |
| | NYGDGISPMAWIGSVDILRRWKEHGCQQVKYGQCWVFAAVACTVLRCL |
| | GIPTRVVTNYNSAHDQNSNLLIEYFRNEYGELESNKSEMIWNFHCWVES |
| | WMTRPDLQPGYEGWQAIDPTPQEKSEGTYCCGPVSVRAIKEGDLSTKY |
| | DASFVFAEVNADVVDWIRQSDGSVLKSINNSLVVGQKISTKSVGRDDRE |
| | DITYTYKYPEGSPEEREVFTRANHLNKLAEKEETGVAMRIRVGDGMSLG |
| | NDFDVFAHIGNDTSESRECRLLLCARTVSYNGVLGPECGTEDINLTLDPY |
| | SENSIPLRILYEKYSGCLTESNLIKVRGLLVEPAANSYLLAER (Rat TG2 |
| | Amino Acid sequence) |
| 43 | DLYLENPEIKIRILGEPKQNRKLVAEVSLKNPLSDSLYDCVFTVEGAGLTK |
| | EQKSVEVSDPVPAGDAVKVRVDLFPTDIGLHKLVVNFQCDKLKSVKGYR |
| | NIIIGPAMAEELLLERCDLEIQANGRDHHTADLCQEKLVLRRGQRFRLTLY |
| | FEGRGYEASVDSLTFGAVTGPDPSEEAGTKARFSLSDNVEEGSWSASV |
| | LDQQDNVLSLQLCTPANAPIGLYRLSLEASTGYQGSSFVLGHFILLYNAW |
| | CPADDVYLDSEEERREYVLTQQGFIYQGSVKFIKSVPWNFGQFEDGILD |
| | TCLMLLDMNPKFLKNRSRDCSRRSSPIYVGRVVSAMVNCNDDQGVLLG |
| | RWDNNYGDGISPMAWIGSVDILRRWKEHGCQQVKYGQCWVFAAVACT |
| | VLRCLGIPTRVVTNYNSAHDQNSNLLIEYFRNEFGELESNKSEMIWNFHC |
| | WESWMTRPDLQPGYEGWQAIDPTPQEKSEGTYCCGPVSVRAIKEGDL |
| | STKYDAPFVFAEVNADVVDWIRQEDGSVLKSINRSLVVGQKISTKSVGRD |
| | DREDITHTYKYPEGSPEEREVFTKANHLNKLAEKEETGVAMRIRVGDSM |
| | SMGNDFDVFAHIGNDTSETRECRLLLCARTVSYNGVLGPECGTEDINLTL |
| | DPYSENSIPLRILYEKYSGCLTESNLIKVRGLLIEPAANSYLLAERDLYLEN |
| | PEIKIRVLGEPKQNRKLVAEVSLKNPLSDPLYDCIFTVEGAGLTKEQKSVE |
| | VSDPVPAGDLVKARVDLFPTDIGLHKLVVNFQCDKLKSVKGYRNVIIGPA |
| | (Mouse TG2 Amino Acid sequence) |

One can easily determine whether an antibody binds to the same epitope as, or competes for binding with, another antibody by using routine methods known in the art. For example, to determine if a test antibody binds to the same epitope as a reference antibody of the invention, the reference antibody is allowed to bind to a protein or peptide under saturating conditions. Next, the ability of a test antibody to bind to the protein or peptide is assessed. If the test antibody is able to bind to the protein or peptide following saturation binding with the reference antibody, it can be concluded that the test antibody binds to a different epitope than the reference antibody. On the other hand, if the test antibody is not able to bind to protein or peptide following saturation binding with the reference antibody, then the test antibody may bind to the same epitope as the epitope bound by the reference antibody of the invention. To determine if an antibody competes for binding with a reference antibody, the above-described binding methodology is performed in two orientations. In a first orientation, the reference antibody is allowed to bind to a protein/peptide under saturating conditions followed by assessment of binding of the test antibody to the protein/peptide molecule. In a second orientation, the test antibody is allowed to bind to the protein/peptide under saturating conditions followed by assessment of binding of the reference antibody to the protein/peptide. If, in both orientations, only the first (saturating) antibody is capable of binding to the protein/peptide, then it is concluded that the test antibody and the reference antibody compete for binding to the protein/peptide. As will be appreciated by the skilled person, an antibody that competes for binding with a reference antibody may not necessarily bind to the identical epitope as the reference antibody, but may sterically block binding of the reference antibody by binding an overlapping or adjacent epitope.

Two antibodies bind to the same or overlapping epitope if each competitively inhibits (blocks) binding of the other to the antigen. That is, a 1-, 5-, 10-, 20- or 100-fold excess of one antibody inhibits binding of the other by at least 50%, 75%, 90% or even 99% as measured in a competitive binding assay. Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

The term “antibody” as used herein includes, but is not limited to, monoclonal antibodies, polyclonal antibodies and recombinant antibodies that are generated by recombinant technologies as known in the art. “Antibody” include antibodies of any species, in particular of mammalian species; such as human antibodies of any isotype, including IgG1, IgG2a, IgG2b, IgG3, IgG4, IgE, IgD and antibodies that are produced as dimers of this basic structure including IgGA1, IgGA2, or pentamers such as IgM and modified variants thereof; non-human primate antibodies, e.g. from chimpanzee, baboon, rhesus or cynomolgus monkey; rodent antibodies, e.g. from mouse, or rat; rabbit, goat or horse antibodies; camelid antibodies (e.g. from camels or llamas such as Nanobodies™) and derivatives thereof; antibodies of bird species such as chicken antibodies; or antibodies of fish species such as shark antibodies.

The term “antibody” also refers to “chimeric” antibodies in which a first portion of at least one heavy and/or light chain antibody sequence is from a first species and a second portion of the heavy and/or light chain antibody sequence is from a second species. Chimeric antibodies of interest herein include “primatized” antibodies comprising variable domain antigen-binding sequences derived from a non-human primate (e.g. Old World Monkey, such as baboon, rhesus or cynomolgus monkey) and human constant region sequences. “Humanized” antibodies are chimeric antibodies that contain a sequence derived from non-human antibodies. For the most part, humanized antibodies are human antibodies (recipient antibody) in which residues from a hypervariable region of the recipient are replaced by residues from a hypervariable region [or complementarity determining region (CDR)] of a non-human species (donor antibody) such as mouse, rat, rabbit, chicken or non-human primate, having the desired specificity, affinity, and activity. In most instances residues of the human (recipient) antibody outside of the CDR; i.e. in the framework region (FR), are additionally replaced by corresponding non-human residues. Furthermore, humanized antibodies may comprise residues that are not found in the recipient antibody or in the donor antibody. These modifications are made to further refine antibody properties. Humanization reduces the immunogenicity of non-human antibodies in humans, thus facilitating the application of antibodies to the treatment of human disease. Humanized antibodies and several different technologies to generate them are well known in the art.

The term “antibody” also refers to human antibodies, which can be generated as an alternative to humanization. For example, it is possible to produce transgenic animals (e.g., mice) that are capable, upon immunization, of producing a full repertoire of human antibodies in the absence of production of endogenous murine antibodies. Other methods for obtaining human antibodies/antibody fragments in vitro are based on display technologies such as phage display or ribosome display technology, wherein recombinant DNA libraries are used that are either generated at least in part artificially or from immunoglobulin variable (V) domain gene repertoires of donors. Phage and ribosome display technologies for generating human antibodies are well known in the art. Human antibodies may also be generated from isolated human B cells that are ex vivo immunized with an antigen of interest and subsequently fused to generate hybridomas which can then be screened for the optimal human antibody.

The term “antibody” refers to both glycosylated and aglycosylated antibodies. Furthermore, the term “antibody” as used herein not only refers to full-length antibodies, but also refers to antibody fragments, more particularly to antigen-binding fragments thereof. A fragment of an antibody comprises at least one heavy or light chain immunoglobulin domain as known in the art and binds to one or more antigen(s). Examples of antibody fragments according to the invention include a Fab, modified Fab, Fab′, modified Fab′, F(ab′)₂, Fv, Fab-Fv, Fab-dsFv, Fab-Fv-Fv, scFv and Bis-scFv fragment. Said fragment can also be a diabody, tribody, triabody, tetrabody, minibody, single domain antibody (dAb) such as sdAb, VL, VH, VHH or camelid antibody (e.g. from camels or llamas such as a Nanobody™) and VNAR fragment. An antigen-binding fragment according to the invention can also comprise a Fab linked to one or two scFvs or dsscFvs, each scFv or dsscFv binding the same or a different target (e.g., one scFv or dsscFv binding a therapeutic target and one scFv or dsscFv that increases half-life by binding, for instance, albumin). Exemplary of such antibody fragments are FabdsscFv (also referred to as BYbe®) or Fab-(dsscFv)2 (also referred to as TrYbe®, see WO2015/197772 for instance). Antibody molecules as defined above, including antigen-binding fragments thereof, are known in the art.

### ASPECTS OF THE INVENTION

1. A method for detecting crosslinks formed by transglutaminases (TGs) in a biological sample, the method comprising the steps of:
a) digesting proteins present in the biological sample using enzymes immobilised on beads, and
b) detecting crosslinks formed by TGs in the biological sample by liquid chromatography-tandem mass spectrometry (LC-MS/MS).

2. A method for determining the activity of transglutaminases (TGs), preferably transglutaminase 2 (TG2), in a subject, the method comprising the steps of:
a) digesting proteins present in a biological sample from the subject using enzymes immobilised on beads, and
b) detecting crosslinks formed by TGs in the biological sample by liquid chromatography-tandem mass spectrometry (LC-MS/MS).

3. The method of aspect 1 or 2, wherein the crosslinks formed by TGs comprise N′N′ bis (γ-glutamyl)-polyamine dipeptides/isopeptides and/or ε-(γ-glutamyl)-lysine dipeptides/isopeptides.

4. The method of any one of aspects 1-3, wherein the method further comprises the step of obtaining the biological sample from a subject.

5. The method of any one of aspects 1-4, wherein the method further comprises the step of enriching the proteins present in the biological sample prior to digestion.

6. The method of any one of aspects 1-5, wherein enriching the proteins comprises precipitating the proteins.

7. The method of aspect 6, wherein enriching the proteins comprises precipitating the proteins by treatment with trichloroacetic acid and separating the precipitated proteins by centrifugation.

8. The method of any one of aspects 1-7, wherein the enzymes immobilised on beads are selected from endopeptidases, exopeptidases, prolidases, or any combination thereof.

9. The method of any one of aspects 1-8, wherein the enzymes immobilised on beads:
(i) are selected from proteinase K, pronase, prolidase, leucine aminopeptidase, carboxypeptidase Y, or any combination thereof; or
(ii) comprise or consist of proteinase K, pronase, prolidase, leucine aminopeptidase and carboxypeptidase Y.

10. The method of any one of aspects 1-9, wherein proteinase K is immobilised on a first population of beads, pronase is immobilised on a second population of beads, prolidase is immobilised on a third population of beads, leucine aminopeptidase is immobilised on a fourth population of beads and carboxypeptidase Y is immobilised on a fifth population of beads.

11. The method of any one of aspects 1-10, wherein the enzymatic digestion of step a) produces a mixture of free amino acids and crosslinks (such as crosslinked dipeptides and/or isopeptides formed by TGs) and wherein said mixture is purified and prepared for LC-MS/MS.

12. The method of any one of aspects 1-11, wherein the crosslinks formed by TGs comprise ε-(γ-glutamyl)-lysine dipeptides.

13. The method of aspect 12, wherein the ε-(γ-glutamyl)-lysine dipeptides are detected using LC-MS/MS as having a retention time equating that of a stable isotope labelled version of the dipeptide and having a parent ion mass/charge ratio (m/z) of 276, and by detecting a range of fragment ion masses derived from the parent ion such as m/z 147, 84 and 130.

14. The method of any one of aspects 1-13, wherein step c) further comprises quantifying the crosslinks formed by TGs by LC-MS/MS.

15. The method of any one of aspects 1-14, the method comprising the steps of:
a) enriching the proteins present in the biological sample by precipitating the proteins by treatment with trichloroacetic acid and separating the precipitated proteins by centrifugation,
b) digesting the proteins using enzymes immobilised on beads, wherein the enzymes comprise proteinase K, pronase, prolidase, leucin aminopeptidase and carboxypeptidase Y, to produce a mixture comprising free amino acids and dipeptides/isopeptides, and
c) detecting and quantifying the amount/number of crosslinks formed by TGs in the biological sample by LC-MS/MS.

16. The method of any one of aspects 1-15, wherein the biological sample is or has been isolated from a subject.

17. The method of any one of aspects 1-16, wherein the biological sample is urine.

18. The method of any one of aspects 1-17, wherein the subject is a mammal, optionally a primate, preferably a human.

19. The method of any one of aspects 1-18, wherein the subject has, or is suspected of having, a disease associated with TG activity, such as TG2 activity.

20. An in vitro method for diagnosing a disease in a subject, the method comprising the steps of:
a) digesting proteins present in the biological sample from the subject using enzymes immobilised on beads, and
b) detecting crosslinks formed by TGs in the biological sample by liquid chromatography-tandem mass spectrometry (LC-MS/MS).

21. An in vitro method for stratifying the severity of a disease in a subject, the method comprising the steps of:
a) digesting proteins present in the biological sample from the subject using enzymes immobilised on beads, and
b) detecting crosslinks formed by TGs in the biological sample by liquid chromatography-tandem mass spectrometry (LC-MS/MS).

22. The method of aspect 20 or 21, wherein the crosslinks formed by TGs comprise N′N′ bis (γ-glutamyl)-polyamine dipeptides/isopeptides and/or ε-(γ-glutamyl)-lysine dipeptides/isopeptides.

23. The method of any one of aspects 20-22, wherein the method further comprises the step of determining the amount/number of crosslinks formed by TGs in the biological sample.

24. The method of any one of aspects 20-23, wherein the presence of crosslinks formed by TGs in the biological sample indicates that the subject has a disease.

25. The method of any one of aspects 20-24, wherein the higher the amount/number of crosslinks formed by TGs in the biological sample the more severe the disease and/or the poorer the prognosis of the disease.

26. The method of any one of aspects 20-25, wherein the disease is any disease associated with elevated TG2 activity, optionally wherein the disease is fibrosis, a fibrotic disease or a fibrosis-related disease such as chronic kidney disease, progressive kidney disease, pulmonary fibrosis, systemic sclerosis, liver cirrhosis, cardiovascular disease, idiopathic hypertrophic cardiomyopathy, renal fibrosis, primary glomerulonephritis, liver cirrhosis, chronic allograft injury (CAI), post-transplant renal fibrosis or chronic allograft nephropathy.

27. An in vitro method for monitoring the progression of a disease in a subject, the method comprising the steps of:
a) digesting proteins present in a biological sample from the subject using enzymes immobilised on beads, and
b) detecting crosslinks formed by TGs in the biological sample by liquid chromatography-tandem mass spectrometry (LC-MS/MS), and
c) comparing the amount/number of crosslinks formed by TGs in the biological sample with the amount/number of crosslinks formed by TGs in a previous sample from the patient or with a control value.

28. The method of aspect 27, wherein the crosslinks formed by TGs comprise N′N′ bis(γ-glutamyl)-polyamine dipeptides/isopeptides and/or ε-(γ-glutamyl)-lysine dipeptides/isopeptides.

29. The method of aspect 27 or 28, wherein the first biological sample was obtained from the subject about 12 months before the second biological sample was obtained from the subject.

30. The method of any one of aspects 27-29, wherein an increase in the amount/number of crosslinks formed by TGs in the second biological sample as compared to the amount/number of crosslinks formed by TGs in (i) the first biological sample, or (ii) the control value, indicates that the disease is worsening.

31. The method of any one of aspects 27-29, wherein a decrease in the amount/number of crosslinks formed by TGs in the second biological sample as compared to the amount/number of crosslinks formed by TGs in (i) the first biological sample, or (ii) the control value, indicates that the disease is improving.

32. The method of any one of aspects 27-31, wherein the disease is any disease associated with elevated TG2 activity, optionally wherein the disease is fibrosis, a fibrotic disease or a fibrosis-related disease such as chronic kidney disease, progressive kidney disease, pulmonary fibrosis, systemic sclerosis, liver cirrhosis, cardiovascular disease, idiopathic hypertrophic cardiomyopathy, renal fibrosis, primary glomerulonephritis, liver cirrhosis, chronic allograft injury (CAI), post-transplant renal fibrosis or chronic allograft nephropathy.

33. A method for determining a subject's response to a treatment, the method comprising the steps of:
a) digesting proteins present in a first biological sample from the subject using enzymes immobilised on beads, and
b) detecting crosslinks formed by TGs in the biological sample by liquid chromatography-tandem mass spectrometry (LC-MS/MS); and
c) comparing the amount/number of crosslinks formed by TGs in the biological sample with the amount/number of crosslinks formed by TGs in a biological sample from the patient prior to treatment, or at an earlier stage in treatment, or with a control value.

34. The method of aspect 33, wherein the crosslinks formed by TGs comprise N′N′ bis(γ-glutamyl)-polyamine dipeptides/isopeptides and/or ε-(γ-glutamyl)-lysine dipeptides/isopeptides.

35. The method of aspect 33 or 34, wherein the method comprises comparing (i) the amount/number of crosslinks formed by TGs in the biological sample, with (ii) the amount/number of crosslinks formed by TGs in a biological sample from a different subject having the disease who has not been administered the treatment; wherein a lower amount/number of crosslinks formed by TGs in (i) as compared to (ii) indicates that the treatment is effective.

36. The method of aspect 33 or 34, wherein the method comprises comparing (i) the amount/number of crosslinks formed by TGs in the biological sample with (ii) the amount/number of crosslinks formed by TGs in a biological sample taken from the subject prior to treatment; wherein a lower amount/number of crosslinks formed by TGs in (i) as compared to (ii) indicates that the treatment is effective.

37. The method of any one of aspects 33-36, wherein the patient is treated with a TG inhibitor.

38. A method for determining the effect of an agent on TG activity, the method comprising the steps of:
a) digesting proteins present in a biological sample from a subject administered the agent using enzymes immobilised on beads, and
b) detecting crosslinks formed by TGs in the biological sample by liquid chromatography-tandem mass spectrometry (LC-MS/MS).

39. The method of aspect 38, wherein the crosslinks formed by TGs comprise N′N′ bis(γ-glutamyl)-polyamine dipeptides/isopeptides and/or ε-(γ-glutamyl)-lysine dipeptides/isopeptides.

40. The method of aspect 38 or 39, wherein the method further comprises comparing (i) the amount/number of crosslinks formed by TGs in the biological sample with (ii) the amount/number of crosslinks formed by TGs in a second biological sample from a different subject who has not been administered the agent; wherein a difference in the amount/number of crosslinks formed by TGs in (i) as compared to (ii) indicates that the agent is having an effect.

41. The method of aspect 38 or 39, wherein the method further comprises comparing (i) the amount/number of crosslinks formed by TGs in the biological sample with (ii) the amount/number of crosslinks formed by TGs in a second biological sample taken from the subject prior to administration of the agent; wherein a difference in the amount/number of crosslinks formed by TGs in (i) as compared to (ii) indicates that the agent is having an effect.

42. The method of any one of aspects 38-41, wherein the difference indicates target engagement by the agent.

43. The method of any one of aspects 38-42, wherein the difference is an increase, which indicates that the agent is a TG activator.

44. The method of any one of aspects 38-43, wherein the difference is a decrease, which indicates that the agent is a TG inhibitor.

45. The method of any one of aspects 38-44, wherein the agent is an anti-TG2 antibody.

### EXAMPLES

### Example 1—Methods

### Urinary Protein Precipitation

Urine samples were collected from patients at different stage of disease advancement and stored at −80° C. until processing. Protein precipitation was performed as follows. Aliquots of urine (4 mL) were added to 1.0 mL of 50% trichloroacetic acid (TCA), vortexed for 10 seconds and centrifuged at 4.200 g for 15 min at 4° C. After removal of the supernatant, the pellet was resuspended in 4 mL of 10% TCA and centrifuged at 4.200 g for 10 min at 4° C. After supernatant removal, the pellet was washed in 4 mL of ethanol:diethyl ether (1:1 v:v) for 3 cycles and in 4 mL of diethyl ether for 2 cycles. After removal of the organic solvents by means of a pipette, the pellet was incubated for 30 min at room temperature to dry. Precipitated proteins were hydrated in 67 μL of 0.1 M potassium phosphate buffer (KPi) containing 6 M urea, pH 7.5 and a bicinchoninic acid (BCA) assay (Pierce, Thermo Scientific) was performed for proteins quantification following the conventional procedure.

### Digestion

Protein digestion was carried out in a thermoblock at 37° C. with 1300 rpm vortexing, using the Enzzen®-Fibrous-Proteins-Digestion kit (Inofea). In more details, precipitated protein solutions were diluted in buffer (0.1 M KPi buffer, pH 7.5, 6M urea) in order to obtain solutions containing 1 mg/mL1 of protein, as measured with the BCA assay. Note that for samples below 1.336 mg/mL, 225 μL of the precipitated protein solution is diluted with 25 μL of the buffer. Upon centrifugation at 2000 g for 1 min at 4° C., 0.05 mL of Enzzen®-Proteinase K was added to a volume of 250 μL of the so-prepared solution and incubated for ˜16 h, at 37° C., 1300 rpm. After 15 min sonication, additional 0.05 mL of Enzzen®-Proteinase K were added and the sample was incubated for 6 h, at 37° C., 1300 rpm. Subsequently, a volume of 1.05 mL of 0.01 M KPi buffer with 0.001 M CaCl₂) at pH 7 and 0.2 mL of Enzzen®-Pronase, 0.05 mL of Enzzen®-Prolidase, 0.1 mL of Enzzen®-Leucin aminopeptidase and 0.1 mL of Enzzen®-Carboxypeptidase Y were added respectively, vortexed for 1 min and samples were incubated for additional 16 h, at 37° C., 1600 rpm. Samples were consequently centrifuged at 2300 g for 15 min at 4° C., and the supernatants were collected and treated for urea removal by means of solid phase extraction using a cation exchange cartridge (Oasis MCX 3 cc Vac Cartridge) as follows. First, the SPE cartridge was conditioned with 1 mL of methanol followed by 1 mL of 2% formic acid (FA). An aliquot of 0.05 mL of the supernatant were added to 0.4 mL of 4% FA, vortexed for 1 min at 1000 rpm and slowly loaded into the cartridge. Successively, the cartridge was washed with 1 mL of 2% FA. The retained charged species were finally eluted twice from the cartridge with 0.5 mL of a solution of methanol and 5% ammonium hydroxide. Samples were consequently vacuum-dried in a SpeedVac system for 12 h. The dried samples were dissolved in 50 μL nanopure water before analysis. To confirm the reproducibility of the digestion and to ensure a linear correlation between the amounts of proteins and of the isopeptide, digestions of fibrous proteins at increasing concentrations were performed in parallel. To confirm the complete digestion of the fibrotic proteins, an amino acid analysis was carried out using the Cell Culture application of the AccQtag Ultra UPLC Amino Acid Solution (Waters Inc.) according to the manufacturer's instructions.

### LC-MS/MS

LC-MS/MS analysis was performed on a Sciex 6500+ triple quadrupole instrument (AB Sciex LLC) coupled with an Acquity I-Class UPLC system (Waters Corporation). Chromatographic separation of ε-(γ-glutamyl) lysine was done on a Waters Acquity BEH Amide column (50×2.1 mm, i.d. 1.7 μm; Waters Corporation) with gradient elution at 50° C. The mobile phase was a mixture of 0.1% (v/v) formic acid in acetonitrile (A) and 0.1% (v/v) formic acid in water (B). After injection 20% B was held for 0.5 min, and two linear gradient steps were programmed, from 20 to 30% B in 0.5 min, and finally from 30 to 80% B in 2.5 min. Then the column was flushed with 80% B for 1.5 min and returned back to 20% B in 0.1 min and finally re-conditioned with 20% B for 1.9 min. Flow rate was maintained constant at 0.4 mL/min. Samples were injected with the partial loop injection mode, weak wash was 600 μL of formic acid:water:acetonitrile (0.1:20:80, v/v/v) and strong wash was 200 μL of formic acid:methanol:water (2:10:90, v/v/v). Flow from the LC was diverted to the MS system from 0.6 to 3.4 min. The MS was fitted with a Turbo Spray Ion Drive source operated in positive electrospray ionization mode. Ion source temperature was set to 500° C. and the ion spray voltage was maintained at 5500 V. Curtain gas, gas 1 and gas 2 pressures were set at 40, 50 and 50 psi, respectively. Collision-induced dissociation was performed with nitrogen gas at the pressure of 9 psi. Selected reaction monitoring with unit mass resolution for the precursor and the product ions was used to quantify the ε-(γ-glutamyl)-lysine dipeptide (Supplementary Table SX). Declustering potential and collision energy were optimized to achieve optimal performance. Data acquisition and processing were carried out by the Analyst software (version 1.6.3; AB Sciex LLC).

### Reported Crosslink Concentration

Formula used to calculate the reported ε-(γ-glutamyl) lysine concentration, by normalizing the measured ε-(γ-glutamyl) lysine concentration in ng/mL to the protein concentration in μg/mL:

$$\begin{matrix}γ - GLU - ε - LYS \\ Concentration \\ ( ng / mg protein )\end{matrix} = \frac{\begin{matrix}Measured γ - GLU - ε - LYS \\ Concentration ( ng / mL )\end{matrix}}{Protein Concentration ( μg / mL )} \times Dilution Factor \times 7400$$

where, ‘Measured γ-GLU-ε-LYS Concentration’ is the concentration of ε-(γ-glutamyl) lysine back-calculated from the calibration curve, ‘Protein Concentration’ is the concentration of the total protein in the reconstituted protein pellet measured by BCA, the ‘Dilution Factor’ accounts for the dilution of reconstituted protein pellet extract subjected to enzymatic digestion and the ‘×7400’ accounts for the dilution of the protein solution (250 μL) that is diluted in a total volume of 1.850 mL to get digested. It is noted that although in this example the measured ε-(γ-glutamyl) lysine concentration (in ng/mL) were normalised to the protein concentration (in μg/mL), other methods well known in the art could be used, such as (but not limited to) normalised to creatine or yet biological sample volume (such as urine volume).

### Example 2—Assay Development

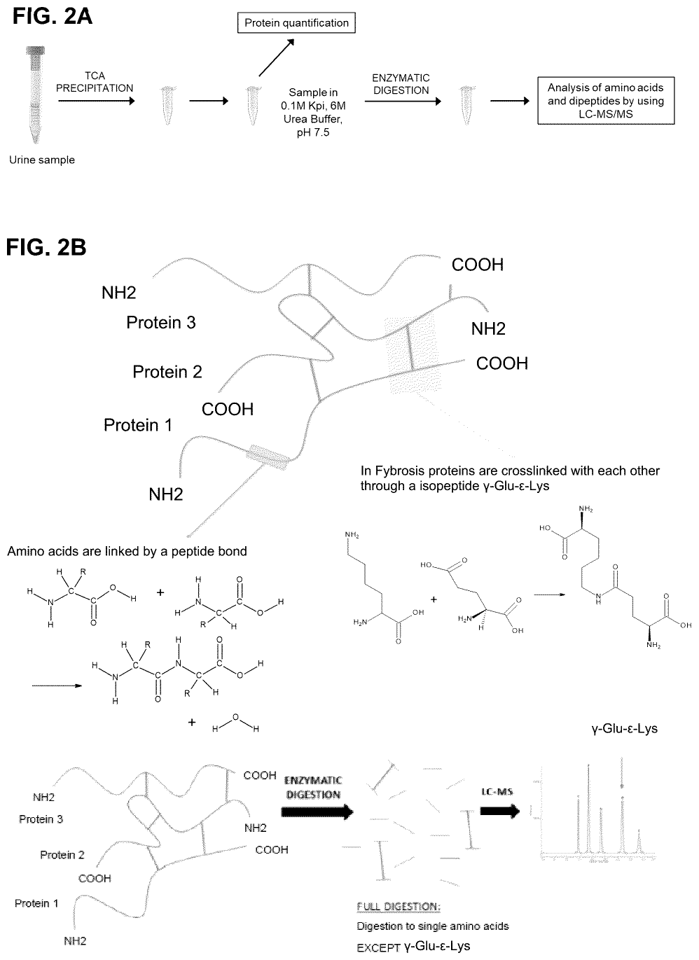

In order to release and quantify ε-(γ-glutamyl) lysine from urinary proteins in an efficient manner, it was necessary to consider several alterations to current methods. The previous methods involve a complex process involving precipitation of the protein from the urine, resuspension of the protein and serial digestion based on a combination of six enzymes, designed to completely digest the diverse array of proteins, but keeping the ε-(γ-glutamyl)-lysine intact. Subsequent detection and quantitation needed to be both sensitive and specific enough for accurate detection of ε-(γ-glutamyl)-lysine (FIG. 2). Several optimisations, compared to previous methods were developed:

### 1. Precipitation Step:

Precipitation, using 4 mL urine, a volume of urine small enough to be handled in reasonable throughput but subsequently shown to provide the required sensitivity. Various methods of precipitation were investigated such as chloroform methanol extraction or trichloroacetic acetic (TCA) (this later was found to be the most effective and reproducible) (See FIG. 2).

### 2. Digestion Step:

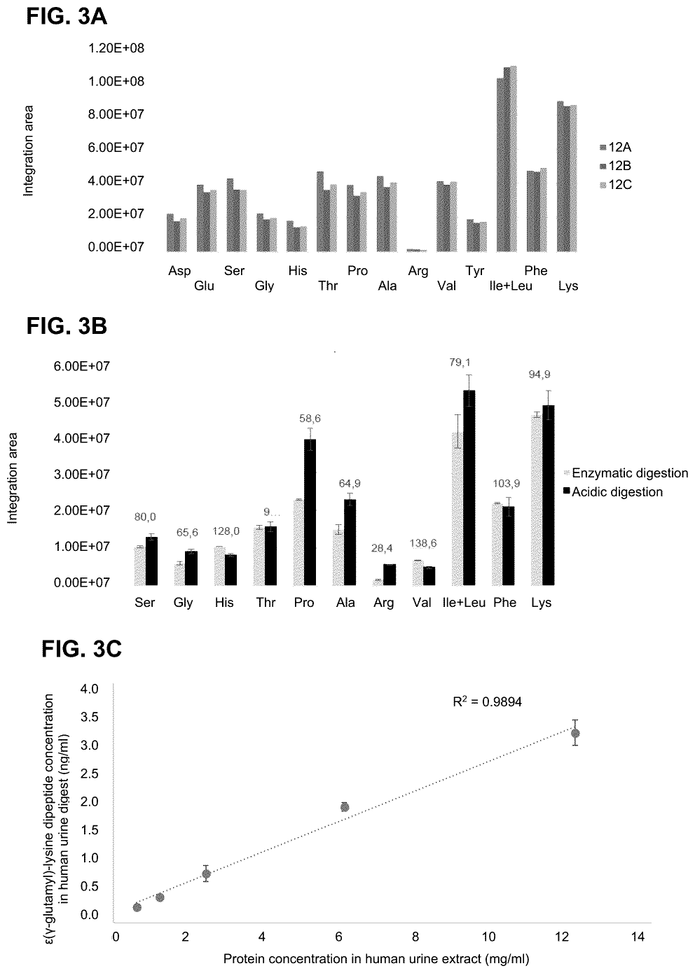

Digestion was optimised and performed using a combination of Proteinase K, Pronase, Prolidase, Aminopeptidase and Carboxypeptidase Y together with the Enzzen® technology. The advantage of such an approach using immobilised enzymes is that the enzymes will be shielded, which prevents them from being digested, and minimises the effect of the enzymes on protein or ε-(γ-glutamyl)-lysine contamination from the reagents. This new digestion step is reproducible, efficient and not impacted by protein content. The reproducibility of the digestion was based on three digestions of the same sample and the CV was <15% (see FIG. 3A). In order to estimate the digestion efficiency, the release of amino acids by the bead digestion was compared to acid hydrolysis, which is considered to be near complete. Such an estimate is hampered by loss of certain amino acids in acid hydrolysis for example, but the digestion efficiency was estimated to be in excess of 80% (see FIG. 3B). To understand the impact of digesting different amount of protein extracted from human urine on the recovery of the ε-(γ-glutamyl) lysine dipeptide, a protein concentration range of 0.6-12 mg/mL was tested. It was demonstrated that there was a strong linear correlation between the protein concentration and the concentration of the ε-(γ-glutamyl)-lysine dipeptide (see FIG. 3C).

### 3. Amino Acid Analysis Step:

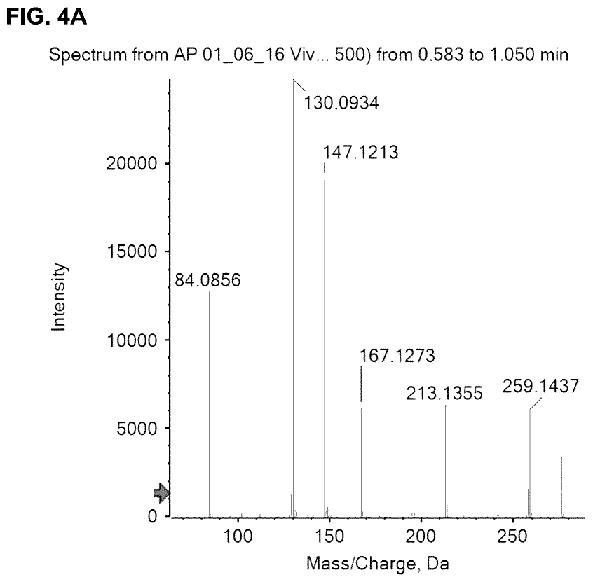

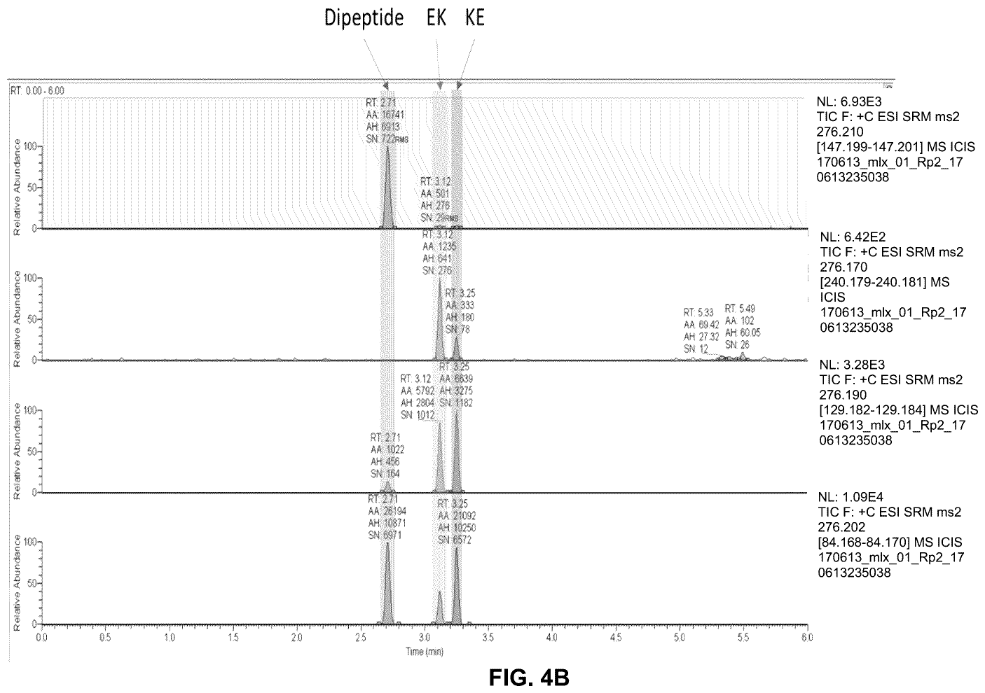

The levels of ε-(γ-glutamyl)-lysine in urine being in the range of ng/mg protein, the peak of ε-(γ-glutamyl)-lysine was extremely low and appeared as an unquantifiable peak in the chromatogram using the classical method. Despite efforts to resolve this issue via the LC parameters, the peaks remained non-integratable. To circumvent this, an alternative method was devised using LC-MS/MS using a triple quadrupole mass spectrometer to aid in the specific detection (see Schafer et al, 2005). Data obtained with such a method showed that it was possible to obtain a mass spectrum for standard ε-(γ-glutamyl)-lysine with characteristic transitions (FIG. 4A) using an ability to resolve the ε-(γ-glutamyl)-lysine peak from any glutamyl-lysine or lysyl-glutamate dipeptide, which may be generated through a partial digestion and release of linear dipeptide from the protein (FIG. 4B). Various chromatographic formats were used (e.g. reverse phase chromatography), but ultimately the best separation and specificity was obtained using a Hydrophilic interaction (HILIC) approach based on an Acquity BEH amide column (Waters), quantifying based on the transitions of 275.9 to 147.1 and 284.2 to 155.2, for analyte and the internal standard, respectively.

Having established the optimised precipitation, digestion and LC-MS/MS parameters, the method was validated for an analytical range of 0.10 ng/mL to 5.00 ng/mL. A linear regression with a 1/×2 weighting was applied to the peak area ratios concentration plot for the construction of calibration curves (data not shown). Representative chromatograms of control blank, zero, LLOQ and ULOQ surrogate matrix sample extracts were obtained (data not shown). The full method showed good intra- and inter-run precision of respectively less than 10% and 20% across the range and sensitivity at 0.1 ng/mL. Moreover, the inter-run accuracy was less than 5%. The precision and accuracy for recombinant ε-(γ-glutamyl)-lysine in surrogate matrix were determined by measurement of ε-(γ-glutamyl)-lysine on 5 occasions at each of four concentrations, each of which were tested in 6 independent replicates per run, resulting in a total of 30 measurements per QC level. In addition, dilutional linearity was assessed, which showed that crosslink levels up to 40 ng/mL were accurately measured in the biological samples.

### Example 3—Testing on Patient Samples

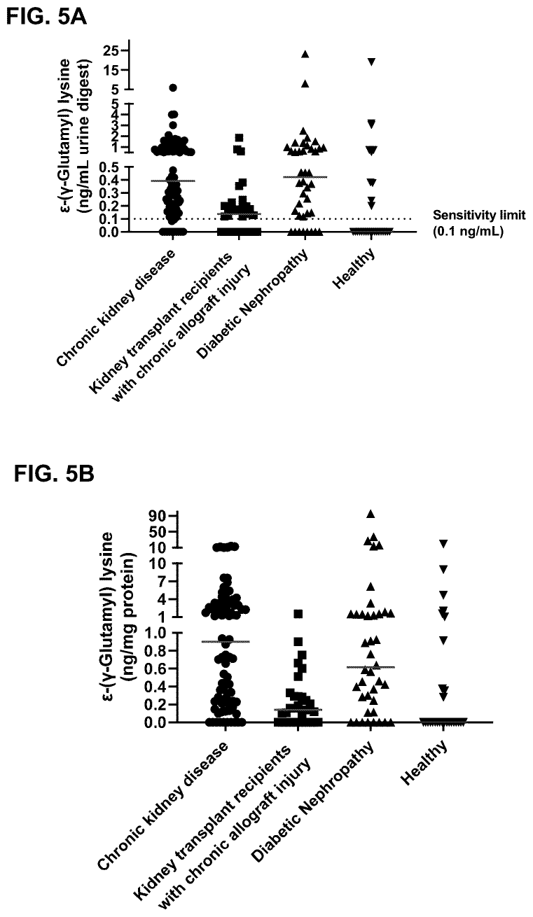

Having established the working method (see example 2), the method applicability was next tested in a range of urine samples from healthy individuals and patients with a variety of disease states. Measurable ε-(γ-glutamyl)-lysine values could be detected in 79% of the disease-state samples whereas in only 46% of the healthy urine samples, the latter of which is mainly due to the low protein content in healthy urine in contrast to the disease-state samples coming from patients with varying high level of proteinuria (FIG. 5A). For urine samples the reported concentration of ε-(γ-glutamyl)-lysine is normalized to mg protein measured in the BCA method and expressed as ng ε-(γ-glutamyl)-lysine equivalent per mg of protein (FIG. 5B). These pre-screened human urine samples were used and mixed to generate endogenous urine QC samples which yield ε-(γ-glutamyl)-lysine at two different concentrations (low and medium). These served to substantiate the method validation with performance evaluation for the endogenous analyte in urine proteins. The precision for determination of ε-(γ-glutamyl)-lysine was determined by measurement of ε-(γ-glutamyl)-lysine and protein in endogenous QC samples on four occasions at each of two concentrations, each of which were tested in six independent replicates per run. Table 1 shows the method presents good intra- and inter-run precision being respectively less than 15% and 25%.

Selectivity was further confirmed during method validation, showing (i) selectivity for the internal standard ε-(γ-Glutamyl)-[U-13C6, 15N2-lysine], assessed in the absence of the internal standard in human urine, as well as in presence of the internal standard in surrogate matrix both in absence and presence (at ULOQ) of ε-(γ-glutamyl)-lysine, and (ii) selectivity for ε-(γ-Glutamyl)-lysine and ε-(γ-Glutamyl)-[U-13C6, 15N2-lysine] in the presence of known ε-(γ-glutamyl)-lysine isomers EK Acid, KE Acid and H-Lys(retro-Glu-H)—OH (Table 1). There were no significant interfering peaks observed at the retention times of ε-(γ-Glutamyl)-lysine and ε-(γ-Glutamyl)-[U-13C6, 15N2-lysine] in the analysed surrogate blank matrix spiked separately with each one of the three isomers (FIG. 4B for EK and KE chromatograms). Additionally, parallelism was successfully demonstrated, by showing a linear concentration response relationship of ε-(γ-glutamyl)-lysine in individual digested human urine samples to the calibration curve (Table 1). Together with the successful parallelism demonstrated in the BCA method (data not shown), it was therefore shown that the method did not suffer from matrix effects. Moreover, ε-(γ-glutamyl)-lysine at the low and medium QC levels was found to be stable in human urine stored in polypropylene containers after 24 hours at the sample processing temperature (room temperature), when stored in a freezer set at −80° C. after 134 days, and after 4 freeze-thaw cycles (at nominally −80° C./room temperature) (Table 1). Finally, carryover was assessed for ε-(γ-glutamyl)-lysine and ε-(γ-Glutamyl)-[U-13C6, 15N2-lysine] in digested surrogate blank matrix and analysed sequentially after the highest calibration standard and was deemed to be acceptable, i.e. no samples where the impact of carryover had the potential to introduce a >15% bias in the measured concentration was identified.

### OVERALL CONCLUSION

A relatively rapid, efficient and selective method for the determination of crosslinks related to TGs activities (N′N′ bis (γ-glutamyl)-polyamine dipeptides/isopeptides and/or ε-(γ-glutamyl)-lysine dipeptides/isopeptides) in human urine is proposed. The method consists of three systematic stages, the first being an efficient and reproducible protein precipitation, which enables the removal of major salts and the concentration of the proteins in a buffer suitable for subsequent digestion. The digestion process has been simplified and streamlined to facilitate complete digestion. Above all, the approach enables digestion of urinary protein without digestion of exogenous proteases and enables the efficient removal of the proteases prior to analysis. The crosslinks were successfully quantified, via LC-MS/MS, from a range of clinical urine samples from patients with a number of diseases. The sensitivity of the method has been shown to be able to detect as low as 0.1 ng/ml of epsilon (gamma-glutamyl) lysine in human urine with coefficient of variation across the entire process of less than 20%. This method represents a significant advance of the previous amino acid analysis-based approach.

### REFERENCES

1. Z. Szondy, et al., BioMedicine (2017) 7:1-13.
2. Schafer et al., J. Agric. Food Chem (2005) 53:2830-2837.
3. WO2015/014888
4. WO2013175229
5. WO2015/197772

TABLE 1
Key characteristics and performance parameters for the assay.
Analytical Methodology

| | |
|---|---|
| Internal standard | ε-(γ-Glutamyl)-[U-13C6, 15N2-lysine] |
| Calibrator and QC | 0.8M urea in 1 mM CaCl₂ and 10 mM |
| surrogate matrix | KPi buffer pH 7.0 |
| Mass spectrometers | Sciex API6500 |
| LC systems | Waters Acquity |
| Sample Preparation | Protein precipitation from urine using |
| Technique | trichloroacetic acid followed by digestion. |
| | Supernatant discarded and pellet washed and |
| | reconstituted in 6M Urea in 0.1M KPi buffer |
| | pH 7.5, before dilution and digestion. |
| | Acetonitrile added to digests before analysis |
| Calibration model | |
| Fit | Linear |
| Weighting | 1/x² |
| Analytical range | 0.100 to 5.00 ng/mL |

Precision and Accuracy Surrogate QC samples

| | | Accuracy |
|---|---|---|
| Surrogate | Precision (% CV) | (% RE) |

| QC samples | Intra-run | Inter-run | Inter-run |
|---|---|---|---|
| LLOQ (0.100 ng/mL) | 7.4% | 16.4% | 3.0% |
| Low QC (0.300 ng/mL)* | 4.9% | 6.9% | −4.0% |
| Med QC (1.00 ng/mL) | 4.0% | 5.8% | −1.8% |
| High QC (3.75 ng/mL) | 3.2% | 4.5% | −2.7% |

Precision endogenous QC samples

| | |
|---|---|
| Endogenous QC samples | Low QC: 3.87 ng/mg |
| (target concentration | Med QC: 10.2 ng/mg |
| in ng/mg protein defined | |
| based on precision runs) | |
| Inter-assay | Low QC: 21.8% |
| precision (% CV) | Med QC: 19.0% |
| Intra-assay | Low QC: 8.8% |
| precision (% CV) | Med QC: 12.5% |

Selectivity

| | |
|---|---|
| In human urine samples | No significant interfering peaks observed |
| | at the retention time of ε-(γ-Glutamyl)-[U- |
| | ¹³C₆, ¹⁵N₂-lysine] in all 10 samples examined |
| For internal standard | Successfully demonstrated in absence and |
| ε-(γ-glutamyl)-[U-¹³C₆, | presence (at ULOQ) of ε-(γ-glutamyl) lysine |
| ¹⁵N₂-lysine] | |
| For ε-(γ-glutamyl) lysine | Successfully demonstrated in |
| in the presence of known | presence of known isomers |
| isomers | |

Matrix effects

| | |
|---|---|
| Parallelism | Successfully demonstrated as more than |
| | 80% of the individual digested human urine |
| | samples met the acceptance criteria with |
| | the back calculated concentration of the |
| | diluted sample <15% of the undiluted |
| | concentration CV ≤15% with |

Stability in human urine

| | |
|---|---|
| Freeze/Thaw | 4 cycles (−80° C./room temperature) |
| Bench top | 24 hours at room temperature |
| Long term | 134 days at nominally −80° C. |
| Carryover | Acceptable; the use of 3 carryover |
| | blank replicates is recommended |

*one value was excluded as identified as outlier.

TABLE 2
SRM table

| | Precursor | Product | Collision | Dwell | Declustering |
|---|---|---|---|---|---|
| Transition | ion | ion | energy | time | potential |
| ID | (m/z) | (m/z) | (V) | (MS) | (V) |
| Analyte 1 | 276 | 147 | 19 | 20 | 46 |
| Analyte 2 ^{a} | | 84 | 39 | | |
| Analyte 3 ^{a} | | 130 | 23 | | |
| SIL-IS 1 ^{a} | 284 | 155 | 19 | | |
| SIL-IS 2 ^{a} | | 90 | 39 | | |
| SIL-IS 3 | | 137 | 23 | | |

^{a} Qualifier transitions for monitoring purposes only, not used in quantitation

1-15. (canceled)

16. A method for detecting crosslinks formed by transglutaminases (TGs) in a biological sample, the method comprising the steps of:
a) digesting proteins present in the biological sample using enzymes immobilised on beads, and
b) detecting crosslinks formed by TGs in the biological sample by liquid chromatography-tandem mass spectrometry (LC-MS/MS).

17. The method of claim 16, wherein:
(i) the crosslinks formed by TGs comprise N′N′ bis (γ-glutamyl)-polyamine dipeptides/isopeptides and/or ε-(γ-glutamyl)-lysine dipeptides/isopeptides;
(ii) the method further comprises the step a0) of obtaining the biological sample from a subject; and/or
(iii) the method further comprises the step a1) of enriching the proteins present in the biological sample prior to digestion; optionally wherein enriching the proteins comprises precipitating the proteins.

18. The method of claim 16, wherein the enzymes immobilised on beads are selected from endopeptidases, exopeptidases, prolidases, or any combination thereof, optionally wherein the enzymes immobilised on beads:
(i) are selected from proteinase K, Pronase, Prolidase, leucine aminopeptidase, Carboxypeptidase Y, or any combination thereof, or
(ii) comprise or consist of proteinase K, Pronase, Prolidase, leucine aminopeptidase and Carboxypeptidase Y.

19. The method of claim 16, wherein:
(i) the enzymatic digestion of step a) produces a mixture of free amino acids and crosslinked dipeptides and/or isopeptides formed by TGs and wherein said mixture is purified and prepared for LC-MS/MS;
(ii) the crosslinks formed by TGs comprise ε-(γ-glutamyl)-lysine dipeptides; optionally wherein the ε-(γ-glutamyl)-lysine dipeptides are detected using LC-MS/MS as having a retention time equating that of a stable isotope labelled version of the dipeptide, and having a parent ion mass/charge ratio (m/z) of 276, and by detecting a range of fragment ion masses derived from the parent ion; and/or
(iii) the method further comprises a step c) of quantifying the crosslinks formed by TGs by LC-MS/MS.

20. The method of claim 16, wherein:
(i) the biological sample is or has been isolated from a subject;
(ii) the biological sample is urine;
(iii) the subject is a mammal, a primate, or a human; and/or
(iv) the subject has, or is suspected of having, a disease associated with TG activity.

21. A method for determining the activity of transglutaminases (TGs) in a subject, the method comprising the steps of:
a) digesting proteins present in a biological sample from the subject using enzymes immobilised on beads, and
b) detecting crosslinks formed by TGs in the biological sample by liquid chromatography-tandem mass spectrometry (LC-MS/MS).

22. The method of claim 21, said method diagnosing a disease in a subject.

23. The method of claim 21, said method stratifying the severity of a disease in the subject.

24. The method of claim 22, wherein:
(i) the crosslinks formed by TGs comprise N′N′ bis (γ-glutamyl)-polyamine dipeptides/isopeptides and/or ε-(γ-glutamyl)-lysine dipeptides/isopeptides;
(ii) the method further comprises the step c) of determining and/or quantifying the amount/number of crosslinks formed by TGs in the biological sample;
(iii) the presence of crosslinks formed by TGs in the biological sample indicates that the subject has a disease;
(iv) the higher the amount/number of crosslinks formed by TGs in the biological sample the more severe the disease and/or the poorer the prognosis of the disease; and/or
(v) the disease is any disease associated with elevated TG activity.

25. The method of claim 24, wherein the disease is fibrosis, a fibrotic disease, a fibrosis-related disease, chronic kidney disease, progressive kidney disease, pulmonary fibrosis, systemic sclerosis, liver cirrhosis, cardiovascular disease, idiopathic hypertrophic cardiomyopathy, renal fibrosis, primary glomerulonephritis, liver cirrhosis, chronic allograft injury (CAI), post-transplant renal fibrosis or chronic allograft nephropathy.

26. An in vitro method for monitoring the progression of a disease in a subject, the method comprising the steps of:
a) digesting proteins present in a biological sample from the subject using enzymes immobilised on beads, and
b) detecting crosslinks formed by TGs in the biological sample by liquid chromatography-tandem mass spectrometry (LC-MS/MS); and
c) comparing the amount/number of crosslinks formed by TGs in the biological sample with the amount/number of crosslinks formed by TGs in a previous sample from the patient or with a control value.

27. The method of claim 26, wherein:
(i) the crosslinks formed by TGs comprise N′N′ bis (γ-glutamyl)-polyamine dipeptides/isopeptides and/or ε-(γ-glutamyl)-lysine dipeptides/isopeptides;
(ii) the first biological sample was obtained from the subject about 12 months before the second biological sample was obtained from the subject;
(iii) an increase in the amount of crosslinks formed by TGs in the second biological sample as compared to the amount of crosslinks formed by TGs in (i) the first biological sample, or (ii) the control value, indicates that the disease is worsening; or a decrease in the amount of crosslinks formed by TGs in the second biological sample as compared to the amount of crosslinks formed by TGs in (i) the first biological sample, or (ii) the control value, indicates that the disease is improving; and/or
(iv) the disease is any disease associated with elevated TG activity.

28. The method of claim 27, wherein the disease is fibrosis, a fibrotic disease, a fibrosis-related disease, chronic kidney disease, progressive kidney disease, pulmonary fibrosis, systemic sclerosis, liver cirrhosis, cardiovascular disease, idiopathic hypertrophic cardiomyopathy, renal fibrosis, primary glomerulonephritis, liver cirrhosis, chronic allograft injury (CAI), post-transplant renal fibrosis or chronic allograft nephropathy.

29. A method for determining a subject's response to a treatment, the method comprising the steps of:
a) digesting proteins present in a first biological sample from the subject using enzymes immobilised on beads, and
b) detecting crosslinks formed by TGs in the biological sample by liquid chromatography-tandem mass spectrometry (LC-MS/MS); and
c) comparing the amount/number of crosslinks formed by TGs in the biological sample with the amount/number of crosslinks formed by TGs in a biological sample from the patient prior to treatment, or at an earlier stage in treatment, or with a control value.

30. The method of claim 29, wherein:
(i) the crosslinks formed by TGs comprise N′N′ bis (γ-glutamyl)-polyamine dipeptides/isopeptides and/or ε-(γ-glutamyl)-lysine dipeptides/isopeptides;
(ii) the method comprises:
a) comparing (i) the amount/number of crosslinks formed by TGs in the biological sample, with (ii) the amount/number of crosslinks formed by TGs in a biological sample from a different subject having the disease who has not been administered the treatment; wherein a lower amount/number of crosslinks formed by TGs in (i) as compared to (ii) indicates that the treatment is effective; or
b) comparing (i) the amount/number of crosslinks formed by TGs in the biological sample with (ii) the amount/number of crosslinks formed by TGs in a biological sample taken from the subject prior to treatment; wherein a lower amount/number of crosslinks formed by TGs in (i) as compared to (ii) indicates that the treatment is effective; and/or
(iii) the patient is treated with an inhibitor of TG.

31. The method of claim 30, wherein the inhibitor of TG is an anti-TG antibody.

32. A method for determining the effect of an agent on TG activity, the method comprising the steps of:
a) digesting proteins present in a biological sample from a subject administered the agent using enzymes immobilised on beads, and
b) detecting crosslinks formed by TGs in the biological sample by liquid chromatography-tandem mass spectrometry (LC-MS/MS).

33. The method of claim 32, wherein:
(i) the crosslinks formed by TGs comprise N′N′ bis (γ-glutamyl)-polyamine dipeptides/isopeptides and/or ε-(γ-glutamyl)-lysine dipeptides/isopeptides;
(ii) the method further comprises the step of:
a) comparing (i) the amount/number of crosslinks formed by TGs in the biological sample with (ii) the amount/number of crosslinks formed by TGs in a second biological sample from a different subject who has not been administered the agent; wherein a difference in the amount/number of crosslinks formed by TGs in (i) as compared to (ii) indicates that the agent is having an effect; or
b) comparing (i) the amount/number of crosslinks formed by TGs in the biological sample with (ii) the amount/number of crosslinks formed by TGs in a second biological sample taken from the subject prior to administration of the agent; wherein a difference in the amount/number of crosslinks formed by TGs in (i) as compared to (ii) indicates that the agent is having an effect;
(iii) the difference indicates target engagement by the agent;
(iv) the difference is an increase, which indicates that the agent is a TG activator; or the difference is a decrease, which indicates that the agent is a TG inhibitor; and/or
(v) the agent is an inhibitor of TG.

34. The method of claim 33, wherein the inhibitor of TG is an anti-TG antibody.


SEQUENCE LISTING (43 sequences)

SEQ ID NO: 1 - protein, 11 aa, synthetic construct
  REGION 1..11: note = L-CDR
KASQDINSYLT

SEQ ID NO: 2 - protein, 7 aa, synthetic construct
  REGION 1..7: note = L-CDR
LVNRLVD

SEQ ID NO: 3 - protein, 9 aa, synthetic construct
  REGION 1..9: note = L-CDR
LQYDDFPYT

SEQ ID NO: 4 - protein, 5 aa, synthetic construct
  REGION 1..5: note = H-CDR
THAMS

SEQ ID NO: 5 - protein, 17 aa, synthetic construct
  REGION 1..17: note = H-CDR
TISSGGRSTYYPDSVKG

SEQ ID NO: 6 - protein, 5 aa, synthetic construct
  REGION 1..5: note = H-CDR
LISTY

SEQ ID NO: 7 - protein, 7 aa, synthetic construct
  REGION 1..7: note = L-CDR
LTNRLMD

SEQ ID NO: 8 - protein, 9 aa, synthetic construct
  REGION 1..9: note = L-CDR
LQYVDFPYT

SEQ ID NO: 9 - protein, 5 aa, synthetic construct
  REGION 1..5: note = H-CDR
SSAMS

SEQ ID NO: 10 - protein, 5 aa, synthetic construct
  REGION 1..5: note = H-CDR
LISPY

SEQ ID NO: 11 - protein, 7 aa, synthetic construct
  REGION 1..7: note = L-CDR
RTNRLFD

SEQ ID NO: 12 - protein, 5 aa, synthetic construct
  REGION 1..5: note = H-CDR
LISLY

SEQ ID NO: 13 - protein, 107 aa, synthetic construct
  REGION 1..107: note = Light Chain
DIQMTQTPSSMYASLGERVTITCKASQDINSYLTWFQQKPGKSPKTLIYRTNRLFDGVPSRFSGSGSGQDFFLTISSLEYEDMGIYYCLQYDDFPYTFGGGTKLEIK

SEQ ID NO: 14 - protein, 107 aa, synthetic construct
  REGION 1..107: note = Light Chain of AB1 antibody
DIKMTQSPSSMYASLGERVTITCKASQDINSYLTWFQQKPGKSPKTLIYRTNRLFDGVPSRFSGSGSGQDFFLTISSLEYEDMGIYYCLQYDDFPYTFGGGTKLEIK

SEQ ID NO: 15 - protein, 107 aa, synthetic construct
  REGION 1..107: note = Light Chain
EIVLTQSPSSMYASLGERVTITCKASQDINSYLTWFQQKPGKSPKTLIYRTNRLFDGVPSRFSGSGSGQDFFLTISSLEYEDMGIYYCLQYDDFPYTFGGGTKLEIK

SEQ ID NO: 16 - protein, 107 aa, synthetic construct
  REGION 1..107: note = Light Chain
DIQMTQSPSSMYASLGERVTITCKASQDINSYLTWFQQKPGKSPKTLIYRTNRLFDGVPSRFSGSGSGQDFFLTISSLEYEDMGIYYCLQYDDFPYTFGGGTKLEIK

SEQ ID NO: 17 - protein, 107 aa, synthetic construct
  REGION 1..107: note = Light Chain
EIVLTQSPSSLSASVGDRVTITCKASQDINSYLTWYQQKPGKAPKLLIYRTNRLFDGVPSRFSGSGSGTDFFFTISSLQPEDFGTYYCLQYDDFPYTFGGGTKLEIK

SEQ ID NO: 18 - protein, 107 aa, synthetic construct
  REGION 1..107: note = Light Chain
DIQMTQSPSSLSASVGDRVTITCKASQDINSYLTWFQQKPGKAPKSLIYRTNRLFDGVPSRFSGSGSGTDFFLTISSLQPEDFATYYCLQYDDFPYTFGQGTKVEIK

SEQ ID NO: 19 - protein, 107 aa, synthetic construct
  REGION 1..107: note = Light Chain of BB7 antibody
AIKMTQSPSSMYASLGERVIITCKASQDINSYLTWFQQKPGKSPKTLIYLTNRLMDGVPSRFSGSGSGQEFLLTISGLEHEDMGIYYCLQYVDFPYTFGGGTKLEIK

SEQ ID NO: 20 - protein, 107 aa, synthetic construct
  REGION 1..107: note = Light Chain
DIQMTQSPSSLSASVGDRVTITCKASQDINSYLTWFQQKPGKAPKSLIYLTNRLMDGVPSRFSGSGSGTDFFLTISSLQPEDFATYYCLQYVDFPYTFGQGTKVEIK

SEQ ID NO: 21 - protein, 107 aa, synthetic construct
  REGION 1..107: note = Light Chain
DIKMTQSPSSLSASVGDRVTITCKASQDINSYLTWFQQKPGKAPKTLIYLTNRLMDGVPSRFSGSGSGQEFLLTISSLQPEDFATYYCLQYVDFPYTFGQGTKVEIK

SEQ ID NO: 22 - protein, 107 aa, synthetic construct
  REGION 1..107: note = Light Chain of DC1 antibody
DITMTQSPSSIYASLGERVTITCKASQDINSYLTWFQQKPGKSPKILIYLVNRLVDGVPSRFSGSGSGQDYALTISSLEYEDMGIYYCLQYDDFPYTFGGGTKLEIK

SEQ ID NO: 23 - protein, 107 aa, synthetic construct
  REGION 1..107: note = Light Chain
DIQMTQSPSSLSASVGDRVTITCKASQDINSYLTWFQQKPGKAPKSLIYLVNRLVDGVPSRFSGSGSGTDFFLTISSLQPEDFATYYCLQYDDFPYTFGQGTKVEIK

SEQ ID NO: 24 - protein, 107 aa, synthetic construct
  REGION 1..107: note = Light Chain of Zampilimab
DITMTQSPSSLSASVGDRVTITCKASQDINSYLTWFQQKPGKAPKILIYLVNRLVDGVPSRFSGSGSGQDYALTISSLQPEDFATYYCLQYDDFPYTFGQGTKVEIK

SEQ ID NO: 25 - protein, 107 aa, synthetic construct
  REGION 1..107: note = Light Chain of rbBB7 antibody
AIKMTQTPSSVSAAVGGTVTISCKASQDINSYLTWFQQKPGQPPKTLIYLTNRLMDGVPSRFSGSGSGQEFLLTISGLECDDAATYYCLQYVDFPYTFGGGTKVVVK

SEQ ID NO: 26 - protein, 112 aa, synthetic construct
  REGION 1..112: note = Light Chain of IA12 antibody
DVVNTQTPLTLSVTFGQPASISCKSSQSLLYDNGKTYLHWLFQRPGQSPRRLIYLVSKLDSGVPDRFTGSGSGTDFTLKISRVEAEDLGVYYCVQGTHFPYTFGGGTKLEIK

SEQ ID NO: 27 - protein, 106 aa, synthetic construct
  REGION 1..106: note = Light Chain
QIVLTQSPAIMSASPGEKVTMTCSASSSVSYMYWYQQKPGSSPRLLIYDTSNLASGVPVRFSGSGSGTSYSLTISRMEAEDAATFYCQQWSSSPLTFGAGTKLELK

SEQ ID NO: 28 - protein, 114 aa, synthetic construct
  REGION 1..114: note = Heavy Chain
EVQLEESGGGLVKPGGSLKLSCAASGFTLSSSAMSWVRQTPDRRLEWVATISVGGGKTYYPDSVKGRFTISRDNAKNTLYLQMNSLRSEDTAMYYCAKLISLYWGQGTTLTVSS

SEQ ID NO: 29 - protein, 114 aa, synthetic construct
  REGION 1..114: note = Heavy Chain of AB1 antibody
EVQLVESGGGLVKPGGSLKLSCAASGFTLSSSAMSWVRQTPDRRLEWVATISVGGGKTYYPDSVKGRFTISRDNAKNTLYLQMNSLRSEDTAMYYCAKLISLYWGQGTTLTVSS

SEQ ID NO: 30 - protein, 114 aa, synthetic construct
  REGION 1..114: note = Heavy Chain
EVQLQESGGGLVKPGGSLKLSCAASGFTLSSSAMSWVRQTPDRRLEWVATISVGGGKTYYPDSVKGRFTISRDNAKNTLYLQMNSLRSEDTAMYYCAKLISLYWGQGTTLTVSS

SEQ ID NO: 31 - protein, 114 aa, synthetic construct
  REGION 1..114: note = Heavy Chain
EVQLLESGGGLVQPGGSLRLSCAASGFTFSSSAMSWVRQAPGKGLEWVSTISVGGGKTYYPDSVKGRFTISRDNSKNTLYLQMNSLRAEDTAVYYCAKLISLYWGQGTLVTVSS

SEQ ID NO: 32 - protein, 114 aa, synthetic construct
  REGION 1..114: note = Heavy Chain of BB7 antibody
AVQLVESGGGLVKPGGSLKLSCAASGIIFSSSAMSWVRQTPEKRLEWVATISSGGRSTYYPDSVKGRFTVSRDSAKNTLYLQMDSLRSEDTAIYYCAKLISPYWGQGTTLTVSS

SEQ ID NO: 33 - protein, 114 aa, synthetic construct
  REGION 1..114: note = Heavy Chain
EVQLLESGGGLVQPGGSLRLSCAASGFTFSSSAMSWVRQAPGKGLEWVSTISSGGRSTYYPDSVKGRFTISRDNSKNTLYLQMNSLRAEDTAVYYCAKLISPYWGQGTLVTVSS

SEQ ID NO: 34 - protein, 114 aa, synthetic construct
  REGION 1..114: note = Heavy Chain
EVQLLESGGGLVQPGGSLRLSCAASGIIFSSSAMSWVRQAPGKGLEWVATISSGGRSTYYPDSVKGRFTVSRDSSKNTLYLQMNSLRAEDTAVYYCAKLISPYWGQGTLVTVSS

SEQ ID NO: 35 - protein, 114 aa, synthetic construct
  REGION 1..114: note = Heavy Chain of DC1 antibody
EVQLVESGGGLVKPGGSLKLSCAASGFTLSTHAMSWVRQTPEKRLEWVATISSGGRSTYYPDSVKGRFTISRDNVKNTLYLQLSSLRSEDTAVYFCARLISTYWGQGTTLTVSS

SEQ ID NO: 36 - protein, 114 aa, synthetic construct
  REGION 1..114: note = Heavy Chain
EVQLLESGGGLVQPGGSLRLSCAASGFTFSTHAMSWVRQAPGKGLEWVSTISSGGRSTYYPDSVKGRFTISRDNSKNTLYLQMNSLRAEDTAVYYCAKLISTYWGQGTLVTVSS

SEQ ID NO: 37 - protein, 114 aa, synthetic construct
  REGION 1..114: note = Heavy Chain of Zampilimab
EVQLLESGGGLVQPGGSLRLSCAASGFTLSTHAMSWVRQAPGKGLEWVATISSGGRSTYYPDSVKGRFTISRDNSKNTLYLQMNSLRAEDTAVYFCARLISTYWGQGTLVTVSS

SEQ ID NO: 38 - protein, 114 aa, synthetic construct
  REGION 1..114: note = Heavy Chain of rbBB7 antibody
EVQLLESGGRLVTPGGSLTLTCAASGIIFSSSAMSWVRQAPGKGLEWVATISSGGRSTYYPDSVKGRFTVSRDSSKNTLYLQMNSLRAEDTAVYYCAKLISPYWGQGTLVTVSS

SEQ ID NO: 39 - protein, 114 aa, synthetic construct
  REGION 1..114: note = Heavy Chain of IA12 antibody
QIQLVQSGPELKKPGETVKISCKASGYTFTTYGNTWVKQAPGKGLKWMGWINTSSGVPTYADDFKGRFAFSLETSASTAYLQINNLKSEDTATYFCARPEVAYWGQGTLVTVSA

SEQ ID NO: 40 - protein, 119 aa, synthetic construct
  REGION 1..119: note = Heavy Chain
QVTLKESGPGILQPSQTLSLTCSFSGFSLSTSGMGVSWIRQPSGKGLEWLAHIYWDDDKRYNPSLKSRLTISKDTSSNQVFLKITSVDTADTATYYCARSGTTAPFAYWGQGTLVTVSA

SEQ ID NO: 41 - protein, 686 aa, Homo sapiens
  REGION 1..686: note = Human TG2
AEELVLERCDLELETNGRDHHTADLCREKLVVRRGQPFWLTLHFEGRNYEASVDSLTFSVVTGPAPSQEAGTKARFPLRDAVEEGDWTATVVDQQDCTLSLQLTTPANAPIGLYRLSLEASTGYQGSSFVLGHFILLFNAWCPADAVYLDSEEERQEYVLTQQGFIYQGSAKFIKNIPWNFGQFEDGILDICLILLDVNPKFLKNAGRDCSRRSSPVYVGRVVSGMVNCNDDQGVLLGRWDNNYGDGVSPMSWIGSVDILRRWKNHGCQRVKYGQCWVFAAVACTVLRCLGIPTRVVTNYNSAHDQNSNLLIEYFRNEFGEIQGDKSEMIWNFHCWVESWMTRPDLQPGYEGWQALDPTPQEKSEGTYCCGPVPVRAIKEGDLSTKYDAPFVFAEVNADVVDWIQQDDGSVHKSINRSLIVGLKISTKSVGRDEREDITHTYKYPEGSSEEREAFTRANHLNKLAEKEETGMAMRIRVGQSMNMGSDFDVFAHITNNTAEEYVCRLLLCARTVSYNGILGPECGTKYLLNLNLEPFSEKSVPLCILYEKYRDCLTESNLIKVRALLVEPVINSYLLAERDLYLENPEIKIRILGEPKQKRKLVAEVSLQNPLPVALEGCTFTVEGAGLTEEQKTVEIPDPVEAGEEVKVRMDLLPLHMGLHKLVVNFESDKLKAVKGFRNVIIGPA

SEQ ID NO: 42 - protein, 579 aa, Rattus rattus
  REGION 1..579: note = Rat TG2
MAEELNLERCDLEIQANGRDHHTADLCQEKLVLRRGQRFRLTLYFEGRGYEASVDRLTFGAVTGPDPSEEAGTKARFSLSDDVEEGSWSASVLDQQDNVLSLQLCTPANAPVGQYRLSLETSTGYQGSSFMLGHFILLFNAWCPADDVYLDSEAERREYVLTQQGFIYQGSVKFIKSVPWNFGQFEDGILDACLMLLDVNPKFLKDRSRDCSRRSSPIYVGRVVSGMVNCNDDQGVLLGRWDNNYGDGISPMAWIGSVDILRRWKEHGCQQVKYGQCWVFAAVACTVLRCLGIPTRVVTNYNSAHDQNSNLLIEYFRNEYGELESNKSEMIWNFHCWVESWMTRPDLQPGYEGWQAIDPTPQEKSEGTYCCGPVSVRAIKEGDLSTKYDASFVFAEVNADVVDWIRQSDGSVLKSINNSLVVGQKISTKSVGRDDREDITYTYKYPEGSPEEREVFTRANHLNKLAEKEETGVAMRIRVGDGMSLGNDFDVFAHIGNDTSESRECRLLLCARTVSYNGVLGPECGTEDINLTLDPYSENSIPLRILYEKYSGCLTESNLIKVRGLLVEPAANSYLLAER

SEQ ID NO: 43 - protein, 793 aa, Mus musculus
  REGION 1..793: note = Mouse TG2
DLYLENPEIKIRILGEPKQNRKLVAEVSLKNPLSDSLYDCVFTVEGAGLTKEQKSVEVSDPVPAGDAVKVRVDLFPTDIGLHKLVVNFQCDKLKSVKGYRNIIIGPAMAEELLLERCDLEIQANGRDHHTADLCQEKLVLRRGQRFRLTLYFEGRGYEASVDSLTFGAVTGPDPSEEAGTKARFSLSDNVEEGSWSASVLDQQDNVLSLQLCTPANAPIGLYRLSLEASTGYQGSSFVLGHFILLYNAWCPADDVYLDSEEERREYVLTQQGFIYQGSVKFIKSVPWNFGQFEDGILDTCLMLLDMNPKFLKNRSRDCSRRSSPIYVGRVVSAMVNCNDDQGVLLGRWDNNYGDGISPMAWIGSVDILRRWKEHGCQQVKYGQCWVFAAVACTVLRCLGIPTRVVTNYNSAHDQNSNLLIEYFRNEFGELESNKSEMIWNFHCWVESWMTRPDLQPGYEGWQAIDPTPQEKSEGTYCCGPVSVRAIKEGDLSTKYDAPFVFAEVNADVVDWIRQEDGSVLKSINRSLVVGQKISTKSVGRDDREDITHTYKYPEGSPEEREVFTKANHLNKLAEKEETGVAMRIRVGDSMSMGNDFDVFAHIGNDTSETRECRLLLCARTVSYNGVLGPECGTEDINLTLDPYSENSIPLRILYEKYSGCLTESNLIKVRGLLIEPAANSYLLAERDLYLENPEIKIRVLGEPKQNRKLVAEVSLKNPLSDPLYDCIFTVEGAGLTKEQKSVEVSDPVPAGDLVKARVDLFPTDIGLHKLVVNFQCDKLKSVKGYRNVIIGPA